(19) United States
(12) Patent Application Publication
(10) Pub. No.: US 2025/0051284 A1
(43) Pub. Date: Feb. 13, 2025

(54) COMPOUNDS FOR THE TREATMENT OF KINASE-DEPENDENT DISORDERS

(71) Applicant: Exelixis, Inc., Alameda, CA (US)

(72) Inventors: Khalid SHAH, Half Moon Bay, CA (US); Frenel DEMORIN, Long Beach, CA (US); Sagar SHAKYA, San Diego, CA (US); Yong WANG, Foster City, CA (US); Wei XU, Danville, CA (US)

(21) Appl. No.: 18/706,757

(22) PCT Filed: Nov. 3, 2022
(86) PCT No.: PCT/US2022/048770

§ 371 Date: May 2, 2024

Related U.S. Application Data
(60) Provisional application No. 63/275,255, filed on Nov. 3, 2021.

(51) Int. Cl. C07D 215/48; A61K 31/47

(52) U.S. Cl. CPC C07D 215/48; A61K 31/47

(57) ABSTRACT

The present disclosure relates to tyrosine kinase inhibitors of Formula I and Compound 1, or pharmaceutically acceptable salts thereof. The present disclosure further relates to processes for the preparation of the tyrosine kinase inhibitors, Formula I and Compound 1, or a pharmaceutically acceptable salt thereof.

### CROSS-REFERENCE TO RELATED APPLICATIONS

This application claims priority to U.S. Application Ser. No. 63/275,255, filed Nov. 3, 2021. The entire contents of the aforementioned application are incorporated herein by reference.

### TECHNICAL FIELD OF THE INVENTION

The present disclosure relates to tyrosine kinase inhibitors of Formula I and Compound 1 that are essentially free of impurities, contaminants, or degradant, or pharmaceutically acceptable salts thereof. The present disclosure further relates to processes for the preparation of the c-Met inhibitors of Formula I and Compound 1, or pharmaceutically acceptable salts thereof.

### BACKGROUND OF THE INVENTION

Protein tyrosine kinases are key regulatory signaling proteins governing cancer cell growth and metastasis. Protein kinase signal transduction is of particular relevance in, for example, renal, gastric, head and neck, lung, breast, prostate, colorectal cancers, and hepatocellular carcinoma; as well as in the growth and proliferation of brain tumor cells.

Small-molecule compounds that specifically inhibit, regulate, and/or modulate the signal transduction of kinases, such as c-Met, VEGFR2, KDR, c-Kit, Axl, flt-3, and flt-4, are desirable as a means to treat or prevent disease states associated with abnormal cell proliferation and angiogenesis. Tyrosine kinase inhibitors (TKIs) have played an increasingly prominent role in the treatment of cancer and other diseases. TKIs with high purity that are free of impurities are especially desirable for use as cancer drugs.

Impurities in a drug may include starting materials, intermediates, byproducts, contaminants, degradation products, and the like. Impurity levels in drugs must be minimized to acceptable safety limits to protect patients. Eliminating or reducing impurities, especially genotoxic impurities, is thus of critical importance. According to the International Council for Harmonization Guidelines (ICH) S2 (R1), genotoxic impurities are broadly defined as impurities that have been demonstrated to cause deleterious changes in the genetic material regardless of the mechanism.

The present invention meets the need of providing kinase inhibitors with high purity and minimal amounts of impurities including genotoxic contaminants. The present invention also meets this need by providing improved processes for making TKIs that minimize the formation of genotoxic contaminants, impurities, byproducts, or degradants.

### SUMMARY OF THE INVENTION

These and other needs are provided by the present invention which is directed to, in one aspect, a compound of formula I.

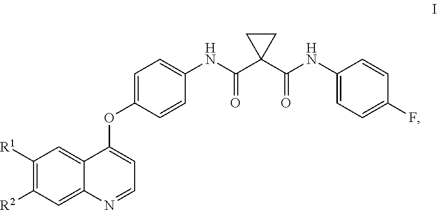

or a pharmaceutically acceptable salt thereof, containing 200 ppm or less of contaminants or degradants including genotoxic contaminants or degradants, wherein:
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl.

Another aspect provides Compound 1

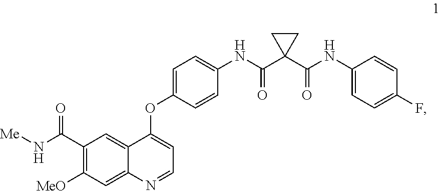 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, comprising 200 ppm or less of contaminants or degradants including genotoxic contaminants or degradants.

Another aspect provides a pharmaceutical composition comprising the compound of formula I or Compound 1, or a pharmaceutically acceptable salt thereof, and one or more

The present invention meets the need of providing kinase inhibitors with high purity and minimal amounts of impurities including genotoxic contaminants. The present invention also meets this need by providing improved processes for making TKIs that minimize the formation of genotoxic contaminants, impurities, byproducts, or degradants.

### SUMMARY OF THE INVENTION

These and other needs are provided by the present invention which is directed to, in one aspect, a compound of formula I:

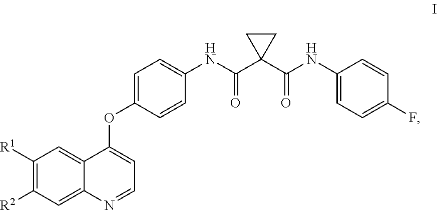

or a pharmaceutically acceptable salt thereof, containing 200 ppm or less of contaminants or degradants including genotoxic contaminants or degradants, wherein:
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl.

Another aspect provides Compound 1

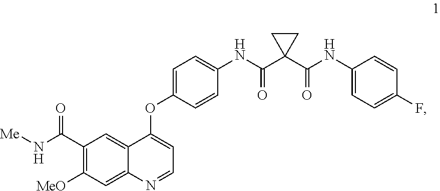 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, comprising 200 ppm or less of contaminants or degradants including genotoxic contaminants or degradants.

Another aspect provides a pharmaceutical composition comprising the compound of formula I or Compound 1, or a pharmaceutically acceptable salt thereof, and one or more pharmaceutically acceptable excipients, wherein the pharmaceutical composition comprises 200 ppm or less of contaminants or degradants including genotoxic contaminants or degradants.

Processes for making the compound of formula I and Compound 1 are also disclosed. Compounds of formula I and Compound 1 are small molecule inhibitors of TAM receptor tyrosine kinases such as Axl and Mer for the treatment of cancer. Compound 1 and its preparation methods are disclosed in PCT application No. PCT/US2019/015297, the entire contents of which are incorporated herein by reference. Compound 1 and its crystalline solid forms and crystalline salts are disclosed in PCT/US2019/065972, the entire contents of which are incorporated herein by reference.

### DETAILED DESCRIPTION OF THE INVENTION

For purposes of this invention, the chemical elements are identified in accordance with the Periodic Table of the Elements, CAS version, Handbook of Chemistry and Physics, 95th Ed. Additionally, general principles of organic chemistry are described in “Organic Chemistry,” 2nd Ed., Thomas Sorrell, University Science Books, Sausalito: 2006, and “March's Advanced Organic Chemistry,” 7th Ed., Ed.: Smith, M. B. and March, J., John Wiley & Sons, New York: 2013, the entire contents of which are hereby incorporated by reference.

As used herein, the term “about” or “approximate” or “approximately” includes (and describes) embodiments that are directed to that value or parameter per se. In certain embodiments, the term “about” or “approximate” or “approximately” includes the indicated amount±10%. In other embodiments, the term “about” approximate” or “approximately” includes the indicated amount±5%. In certain other embodiments, the term “about” approximate” or “approximately” includes the indicated amount±1%.

As used herein, the term “slurry” refers to a mixture of solids suspended in liquid. It can be prepared by adding enough solids to a given solvent at ambient conditions so that undissolved solids are present. It can be prepared by agitation (typically by stirring or oscillation), an act that is also referred to as “slurrying,” in a sealed vial at a given temperature for an extended period of time. Typically, the solids are recovered after a given period of time using a method described herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit risk ratio.

As used herein, the term “catalytic amount” means an amount that is less than a stoichiometric equivalent of the limiting reagent. In some embodiments, a catalytic amount is much less than a stoichiometric equivalent of the limiting reagent, for example, between 0 and 5 weight percent (wt %), between 0 and 4 wt %, between 0 and 3 wt %, between 0 and 2 wt %, between 0 and 1 wt %, between 0 and 0.9 wt %, between 0 and 0.8 wt %, between 0 and 0.7 wt %, between 0 and 0.6 wt %, between 0 and 0.5 wt %, between 0 and 0.4 wt %, between 0 and 0.3 wt %, between 0 and 0.2 wt %, between 0 and 0.1 wt %, between 0 and 0.05 wt %, and between 0 and 0.01 wt % of the stoichiometric amount of the limiting reagent.

The phrase “essentially free” as used in the phrase “essentially free of contaminants, degradants, or impurities,” means that a compound or composition as disclosed herein is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 3 ppm or less, 2.5 ppm or less, or 1 ppm or less of such contaminants, degradants, or impurities.

In general, the nomenclature used in this application is based on naming conventions adopted by the international union of pure and applied chemistry (IUPAC). Chemical structures shown herein were prepared using CHEMDRAW®. Any open valency appearing on a carbon, oxygen, or nitrogen atom in the structures herein indicates the presence of a hydrogen atom.

Examples of pharmaceutically acceptable excipients, diluents, fillers, binders, disintegrants, glidants, lubricants, and coatings are described in more detail in references readily available to the skilled practitioner, for instance, in the Handbook of Pharmaceutical Excipients, 9^{th} Ed., Paul J Sheskey, Bruno C Hancock, Gary P Moss, David J Goldfarb, 2020; and Remington, The Science and Practice of Pharmacy, 23^{rd} Ed., Adeboye Adejare, Philadelphia, PA.

The diluent may be any diluent known to a person of ordinary skill in the art. In one embodiment, the diluent is an inorganic diluent, polysaccharide, mono- or disaccharide or sugar alcohol. In another embodiment, the diluent comprises lactose, microcrystalline cellulose, starch, corn starch, croscarmellose sodium, or a mixture thereof.

The filler may be any filler known to a person of ordinary skill in the art. Examples of fillers include lactose, microcrystalline cellulose, starch, corn starch, croscarmellose sodium, sucrose, calcium phosphate, maltodextrin, mannitol, inorganic salts, and mixtures thereof.

The binder may be any binder known to a person of ordinary skill in the art. Suitable binders comprises sodium carboxymethylcellulose, polyvinyl pyrrolidone (PVP), copovidone, polyvinyl pyrrolidone-vinyl acetate (PVP/VA) copolymer, hydroxypropylcellulose, hydroxypropyl methylcellulose, ethyl cellulose, or a mixture thereof.

The disintegrant may be any disintegrant known to a person of ordinary skill in the art.

Suitable disintegrants comprises croscarmellose sodium, crospovidone, low-substituted hydroxypropylcellulose, sodium starch glycolate, or a mixture thereof.

The glidant may be any glidant known to a person of ordinary skill in the art. Suitable glidants include starch, corn starch, silicon dioxide, colloidal silicon dioxide, or a mixture thereof. In another embodiment, the glidant is silicon dioxide.

The lubricant may be any lubricant known to a person of ordinary skill in the art. In another embodiment, the lubricant is stearic acid or magnesium stearate.

The film coating relates to a mixture of pharmaceutically acceptable excipients that are typically applied to a compressed tablet, beads, granules, or particles of active ingredient that are compressed into tablets. It is understood that the coating chosen must be compatible with the active agent. It is further understood that a person skilled in the art will know how to manipulate the coating to achieve disintegration in the stomach by choosing the excipients which make up the coating, its type, and/or its thickness.

Suitable polymers for film-coating are soluble at pH of from about 1.2 to about 5, such as for example hydroxypropylmethylcellulose (HPMC) alone and/or in combination with hydroxypropylcellulose (HPC), carboxymethylcellulose, methylcellulose, ethylcellulose, acrylic resins, and polyvinylpyrrolidone and gelatin or other commercially available film-coating preparations such as Dri-Klear® (Crompton & Knowles Corp., Mahwah, N.J.) or Opadry® (Colorcon, West Point Pa.).

The symbol “—” means a single bond, and “═” means a double bond.

As used herein, the singular forms “a”, “an”, and “the” include plural reference unless the context clearly dictates otherwise.

When a variable is defined generically, with a number of possible substituents, each individual radical can be defined with or without the bond. For example, if R^{z} can be hydrogen, this can be indicated as “—H” or “H” in the definition of R^{z}.

### Embodiments

In one aspect, the present disclosure provides a compound of formula I:

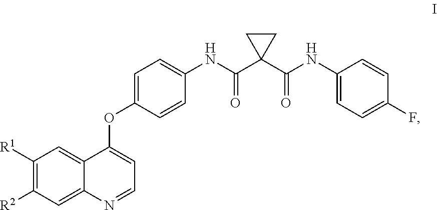

or a pharmaceutically acceptable salt thereof, comprising 200 ppm or less contaminants or degradants, wherein:
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl.

In one aspect, the present disclosure provides a compound of formula I:

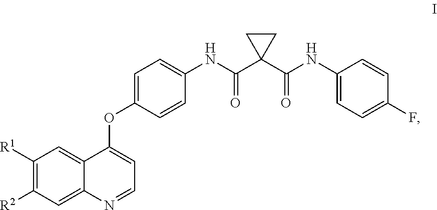

or a pharmaceutically acceptable salt thereof, comprising 200 ppm or less genotoxic contaminants or degradants, wherein:
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl.

In one aspect, the present disclosure provides a compound of formula I:

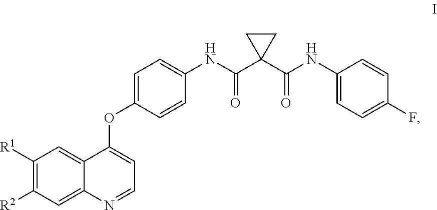

or a pharmaceutically acceptable salt thereof, comprising 100 ppm or less contaminants or degradants, wherein:
R¹ is —C(O)—NH₂, —C(O)—NH(C₁₋₆ alkyl), or —C(O)—N(C₁₋₆ alkyl)₂; and
R² is —OC₁₋₆ alkyl.

In one aspect, the present disclosure provides a compound of formula I:

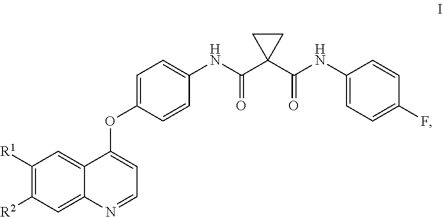

or a pharmaceutically acceptable salt thereof, comprising 100 ppm or less genotoxic contaminants or degradants, wherein:
R¹ is —C(O)—NH₂, —C(O)—NH(C₁₋₆ alkyl), or —C(O)—N(C₁₋₆ alkyl)₂; and
R² is —OC₁₋₆ alkyl.

In some embodiments, the contaminants or degradants comprise 4-aminophenol, 4-fluoroaniline,

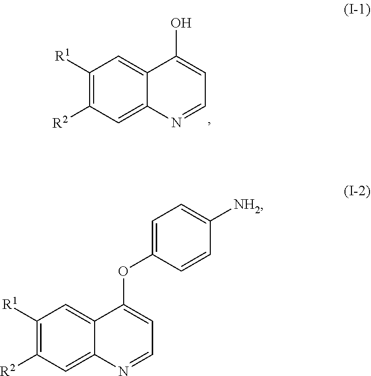

or mixtures thereof.

The contaminants or degradants comprise genotoxic contaminants or degradants.

In some embodiments, the genotoxic contaminants or degradants comprise 4-aminophenol, 4-fluoroaniline,

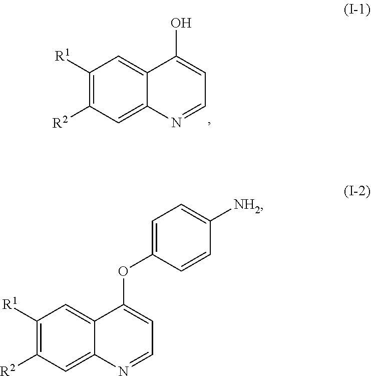

or mixtures thereof.

In some embodiments, the genotoxic contaminants or degradants comprise 4-aminophenol, 4-fluoroaniline,

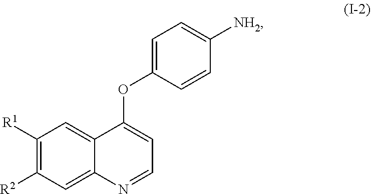

or mixtures thereof.

In one embodiment, the present disclosure provides a compound of formula I:

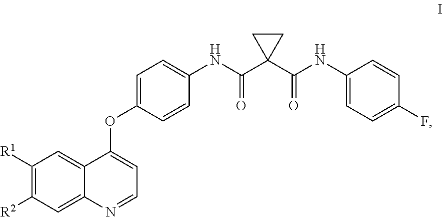

or a pharmaceutically acceptable salt thereof, comprising 200 ppm or less contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl.
wherein the contaminants or degradants comprise 4-aminophenol, 4-fluoroaniline, NH₂

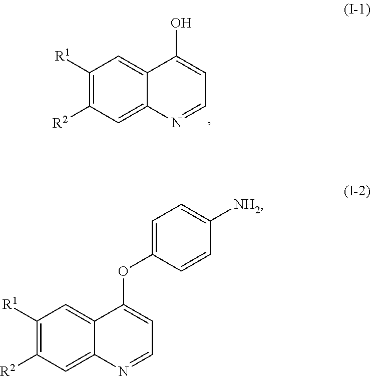

or mixtures thereof.

In one embodiment, the present disclosure provides a compound of formula I:

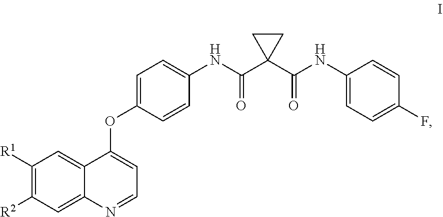

or a pharmaceutically acceptable salt thereof, comprising 100 ppm or less of genotoxic contaminants or degradants, wherein
R¹ is —C(O)—NH₂, —C(O)—NH(C₁₋₆ alkyl), or —C(O)—N(C₁₋₆ alkyl)₂; and
R² is —OC₁₋₆ alkyl.
   wherein the genotoxic contaminants or degradants comprise 4-aminophenol, 4-fluoroaniline,

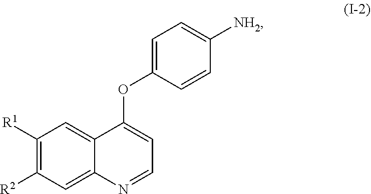

or mixtures thereof.

In one aspect, the present disclosure provides a pharmaceutical composition comprising the compound of formula I

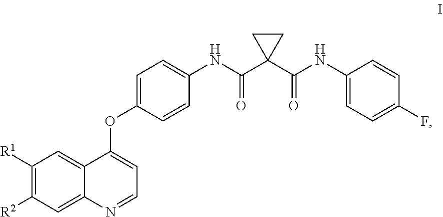

or a pharmaceutically acceptable salt thereof, and one or more pharmaceutically acceptable excipients, wherein the pharmaceutical composition comprises 200 ppm or less of contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl.

In one embodiment, the present disclosure provides a pharmaceutical composition comprising the compound of formula I:

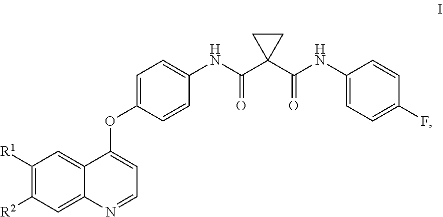

or a pharmaceutically acceptable salt thereof, and one or more pharmaceutically acceptable excipients, wherein the pharmaceutical composition comprises 200 ppm or less of contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl,
wherein the contaminants or degradants comprise 4-aminophenol, 4-fluoroaniline,

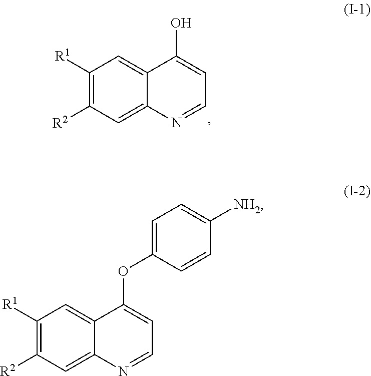

or mixtures thereof.

In some embodiments, the one or more pharmaceutically acceptable excipients comprise one or more fillers; one or more disintegrants; one or more glidants; and one or more lubricants.

In some embodiments, the one or more pharmaceutically acceptable excipients comprise one or more diluent; one or more disintegrants; one or more glidants; one or more binder; and one or more lubricants.

In one embodiment, the pharmaceutical composition is a tablet.

In another embodiment, the pharmaceutical composition is a capsule.

In some embodiments, R¹ is —COOH or —C(O)—NHMe.

In some embodiments, R¹ is —C(O)—NHMe.

In some embodiments, R² is —OMe.

In some embodiments, R¹ is —C(O)—NHMe and R² is —OMe.

In some embodiments, the contaminants or degradants are 100 ppm or less, 75 ppm or less, 50 ppm or less, 25 ppm or less, 20 ppm or less, 15 ppm or less, 13 ppm or less, 10 ppm or less, 5 ppm or less, 3 ppm or less, 2.5 ppm or less, or 1 ppm or less.

In some embodiments, the genotoxic contaminants or degradants are 100 ppm or less, 75 ppm or less, 50 ppm or less, 25 ppm or less, 20 ppm or less, 15 ppm or less, 13 ppm or less, 10 ppm or less, 5 ppm or less, 3 ppm or less, 2.5 ppm or less, or 1 ppm or less.

In some embodiments, the genotoxic contaminants or degradants are 75 ppm or less, 50 ppm or less, 25 ppm or less, 20 ppm or less, 15 ppm or less, 13 ppm or less, 10 ppm or less, 5 ppm or less, or 2.5 ppm or less, or 1 ppm or less.

In some embodiments, the compound of formula I or the pharmaceutically acceptable salt thereof is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less impurities, contaminants, or degradants.

In some embodiments, the compound of formula I or the pharmaceutically acceptable salt thereof is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less impurities, contaminants, or degradants comprising 4-aminophenol, 4-fluoroaniline, (I-1), (I-2), or mixtures thereof.

In some embodiments, the compound of formula I or the pharmaceutically acceptable salt thereof is mixed with 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less 4-aminophenol.

In some embodiments, the compound of formula I or the pharmaceutically acceptable salt thereof is mixed with 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less 4-fluoroaniline.

In some embodiments, the compound of formula I or the pharmaceutically acceptable salt thereof is mixed with 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less the compound of formula I-1.

In some embodiments, the compound of formula I or the pharmaceutically acceptable salt thereof is mixed with 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less the compound of formula I-1.

In some embodiments, the compound of formula I or the pharmaceutically acceptable salt thereof is mixed with 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less the compound of formula I-2.

In some embodiments, the compound of formula I or the pharmaceutically acceptable salt thereof is mixed with at least two of 4-aminophenol, 4-fluoroaniline, (I-1), and (I-2), wherein the combined level of the at least two of 4-aminophenol, 4-fluoroaniline, (I-1), and (I-2) is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less.

In some embodiments, the compound of formula I or the pharmaceutically acceptable salt thereof is mixed with at least three of 4-aminophenol, 4-fluoroaniline, (I-1), and (I-2), wherein the combined level of the at least three of 4-aminophenol, 4-fluoroaniline, (I-1), and (I-2) is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less.

In some embodiments, the compound of formula I or the pharmaceutically acceptable salt thereof is mixed with 4-aminophenol, 4-fluoroaniline, (I-1), and (I-2), wherein the combined level of 4-aminophenol, 4-fluoroaniline, (I-1), and (I-2) is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less.

In some embodiments, the compound of formula I or the pharmaceutically acceptable salt thereof is mixed with 4-fluoroaniline and the compound of formula I-2, and the combined level of 4-fluoroaniline and the compound of formula I-2 is 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less.

In some embodiments, the compound of formula I or the pharmaceutically acceptable salt thereof is mixed with 4-aminophenol and the compound of formula I-2, and the combined level of 4-aminophenol and the compound of formula I-2 is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less.

In some embodiments, the compound of formula I or the pharmaceutically acceptable salt thereof is mixed with the compound of formula I-1 and the compound of formula I-2, and the combined level of the compounds of formulas I-1 an I-2 is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less.

Another aspect of the present disclosure provides Compound 1

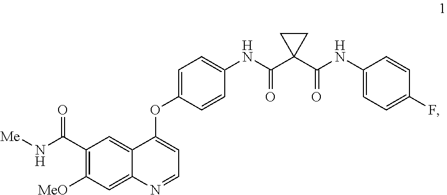 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, comprising 200 ppm or less of contaminants or degradants.

Another aspect of the present disclosure provides Compound 1

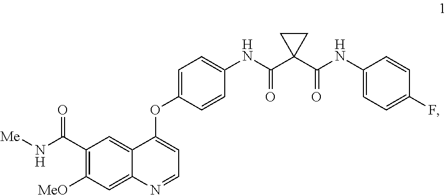 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, comprising 100 ppm or less of contaminants or degradants.

Another aspect of the present disclosure provides Compound 1

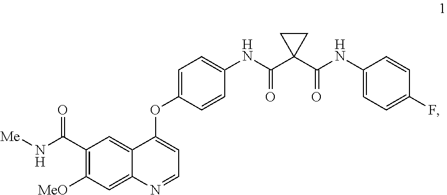 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, comprising 200 ppm or less of genotoxic contaminants or degradants.

Another aspect of the present disclosure provides Compound 1

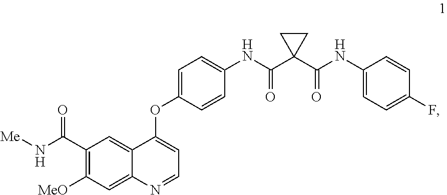 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, comprising 100 ppm or less of genotoxic contaminants or degradants.

Another aspect of the present disclosure provides a pharmaceutical composition comprising Compound 1 or a pharmaceutically acceptable salt thereof, and one or more pharmaceutically acceptable excipients, wherein the pharmaceutical composition comprises 200 ppm or less of contaminants or degradants.

In some embodiments, the one or more pharmaceutically acceptable excipients comprise one or more fillers; one or more disintegrants; one or more glidants; and one or more lubricants.

In some embodiments, the one or more pharmaceutically acceptable excipients comprise one or more diluent; one or more disintegrants; one or more glidants; one or more binder; and one or more lubricants.

In one embodiment, the pharmaceutical composition is a tablet.

In another embodiment, the pharmaceutical composition is a capsule.

In some embodiments, the contaminants or degradants comprise 4-aminophenol, 4-fluoroaniline,

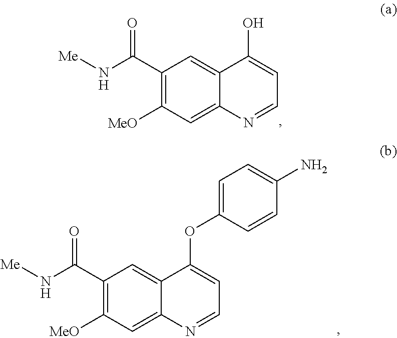

or mixtures thereof.

In some embodiments, the contaminants or degradants comprise genotoxic contaminants or degradants.

In some embodiments, the genotoxic contaminants or degradants comprise 4-aminophenol, 4-fluoroaniline,

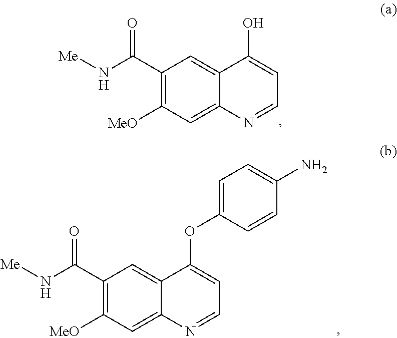

or mixtures thereof.

In some embodiments, the genotoxic contaminants or degradants comprise 4-aminophenol, 4-fluoroaniline, or mixtures thereof.

In some embodiments, the genotoxic contaminants or degradants comprise 4-fluoroaniline.

4-Aminophenol, 4-fluoroaniline, Compound a, and Compound b are starting materials or intermediates used in the synthesis of Compound 1 and its pharmaceutically acceptable salt, or by-products or degradants produced in the process, or impurities from the starting materials. These compounds are also possible genotoxic impurities that are undesirable in the final drug product. Thus, it is important to eliminate or minimize the level of these impurities or other genotoxic impurities. The presence of low levels of these impurities is difficult to avoid because no other viable synthetic routes have been identified that do not include these materials. The present disclosure provides improved manufacturing processes to ensure that the levels of these impurities are as low as reasonably possible.

In some embodiments, the contaminants or degradants are 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 3 ppm or less, 2.5 ppm or less, or 1 ppm or less.

In some embodiments, the genotoxic contaminants or degradants are 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 3 ppm or less, 2.5 ppm or less, or 1 ppm or less.

In some embodiments, the contaminants or degradants, or genotoxic contaminants or degradants, are, including the endpoints, 200 ppm to 0.001 ppm, 100 ppm to 0.001 ppm, 75 ppm to 0.001 ppm, 50 ppm to 0.001 ppm, 25 ppm to 0.001 ppm, 20 ppm to 0.001 ppm, 15 ppm to 0.001 ppm, 13 ppm to 0.001 ppm, 10 ppm to 0.001 ppm, 5 ppm to 0.001 ppm, 3 ppm to 0.001 ppm, 2.5 ppm to 0.001 ppm, 2 ppm to 0.001 ppm, or 1 ppm to 0.001 ppm.

In some embodiments, the contaminants or degradants, or genotoxic contaminants or degradants, are, including the endpoints, 200 ppm to 0.01 ppm, 100 ppm to 0.01 ppm, 75 ppm to 0.01 ppm, 50 ppm to 0.01 ppm, 25 ppm to 0.01 ppm, 20 ppm to 0.01 ppm, 15 ppm to 0.01 ppm, 13 ppm to 0.01 ppm, 10 ppm to 0.01 ppm, 5 ppm to 0.01 ppm, 3 ppm to 0.01 ppm, 2.5 ppm to 0.01 ppm, 2 ppm to 0.01 ppm, or 1 ppm to 0.01 ppm.

In some embodiments, the contaminants or degradants, or genotoxic contaminants or degradants, are, including the endpoints, 200 ppm to 0.1 ppm, 100 ppm to 0.1 ppm, 75 ppm to 0.1 ppm, 50 ppm to 0.1 ppm, 25 ppm to 0.1 ppm, 20 ppm to 0.1 ppm, 15 ppm to 0.1 ppm, 13 ppm to 0.1 ppm, 10 ppm to 0.1 ppm, 5 ppm to 0.1 ppm, 3 ppm to 0.1 ppm, 2.5 ppm to 0.1 ppm, 2 ppm to 0.1 ppm, or 1 ppm to 0.1 ppm.

In some embodiments, the pharmaceutically acceptable salt is Compound 1 hemifumarate.

In some embodiments, Compound 1 freebase or Compound 1 hemifumarate is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less impurities, contaminants, or degradants.

In some embodiments, Compound 1 freebase or Compound 1 hemifumarate is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less genotoxic impurities, contaminants, or degradants.

In some embodiments, Compound 1 freebase or Compound 1 hemifumarate is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less impurities, contaminants, or degradants comprising 4-aminophenol, 4-fluoroaniline, compound a, Compound b, or mixtures thereof.

In some embodiments, Compound 1 freebase or Compound 1 hemifumarate is mixed with 75 ppm or less, 50 ppm or less, 25 ppm or less, 20 ppm or less, 15 ppm or less, 13 ppm or less, 10 ppm or less, 5 ppm or less, or 2.5 ppm or less 4-aminophenol, 4-fluoroaniline, Compound a, Compound b, or mixtures thereof.

In some embodiments, Compound 1 freebase or Compound 1 hemifumarate is mixed with, including the endpoints, 200 ppm to 0.001 ppm, 100 ppm to 0.001 ppm, 75 ppm to 0.001 ppm, 50 ppm to 0.001 ppm, 25 ppm to 0.001 ppm, 20 ppm to 0.001 ppm, 15 ppm to 0.001 ppm, 13 ppm to 0.001 ppm, 10 ppm to 0.001 ppm, 5 ppm to 0.001 ppm, 3 ppm to 0.001 ppm, 2.5 ppm to 0.001 ppm, 2 ppm to 0.001 ppm, or 1 ppm to 0.001 ppm 4-aminophenol, 4-fluoroaniline, Compound a, Compound b, or mixtures thereof.

In some embodiments, Compound 1 freebase or Compound 1 hemifumarate is mixed with, including the endpoints, 200 ppm to 0.01 ppm, 100 ppm to 0.01 ppm, 75 ppm to 0.01 ppm, 50 ppm to 0.01 ppm, 25 ppm to 0.01 ppm, 20 ppm to 0.01 ppm, 15 ppm to 0.01 ppm, 13 ppm to 0.01 ppm, 10 ppm to 0.01 ppm, 5 ppm to 0.01 ppm, 3 ppm to 0.01 ppm, 2.5 ppm to 0.01 ppm, 2 ppm to 0.01 ppm, or 1 ppm to 0.01 ppm 4-aminophenol, 4-fluoroaniline, Compound a, Compound b, or mixtures thereof.

In some embodiments Compound 1 freebase or Compound 1 hemifumarate is mixed with, including the endpoints, 200 ppm to 0.1 ppm, 100 ppm to 0.1 ppm, 75 ppm to 0.1 ppm, 50 ppm to 0.1 ppm, 25 ppm to 0.1 ppm, 20 ppm to 0.1 ppm, 15 ppm to 0.1 ppm, 13 ppm to 0.1 ppm, 10 ppm to 0.1 ppm, 5 ppm to 0.1 ppm, 3 ppm to 0.1 ppm, 2.5 ppm to 0.1 ppm, 2 ppm to 0.1 ppm, or 1 ppm to 0.1 ppm 4-aminophenol, 4-fluoroaniline, Compound a, Compound b, or mixtures thereof.

In some embodiments, the contaminants or genotoxic contaminants or degradants comprise 4-aminophenol.

In some embodiments, Compound 1 freebase or Compound 1 hemifumarate is mixed with 100 ppm or less, 75 ppm or less, 50 ppm or less, 25 ppm or less, 20 ppm or less, 15 ppm or less, 13 ppm or less, 10 ppm or less, 5 ppm or less, 3 ppm or less, 2.5 ppm or less, or 1 ppm or less 4-aminophenol.

In some embodiments, Compound 1 freebase or Compound 1 hemifumarate is mixed with 75 ppm or less, 50 ppm or less, 25 ppm or less, 20 ppm or less, 15 ppm or less, 13 ppm or less, 10 ppm or less, 5 ppm or less, or 2.5 ppm or less 4-aminophenol.

In some embodiments, Compound 1 freebase is mixed with 15 ppm or less 4-aminophenol.

In some embodiments, Compound 1 hemifumarate is mixed with 15 ppm or less 4-aminophenol.

In some embodiments, Compound 1 freebase is mixed with 3 ppm or less 4-aminophenol.

In some embodiments, Compound 1 hemifumarate is mixed with 3 ppm or less 4-aminophenol.

In some embodiments, the contaminants or genotoxic contaminants or degradants comprise 4-fluoroaniline.

In some embodiments, Compound 1 freebase or Compound 1 hemifumarate is mixed with 75 ppm or less, 50 ppm or less, 25 ppm or less, 20 ppm or less, 15 ppm or less, 13 ppm or less, 10 ppm or less, 5 ppm or less, or 2.5 ppm or less, or 1 ppm or less 4-fluoroaniline.

In some embodiments, Compound 1 freebase is mixed with 15 ppm or less 4-fluoroaniline.

In some embodiments, Compound 1 hemifumarate is mixed with 15 ppm or less 4-fluoroaniline.

In some embodiments, Compound 1 freebase is mixed with 5 ppm or less 4-fluoroaniline.

In some embodiments, Compound 1 hemifumarate is mixed with 5 ppm or less 4-fluoroaniline.

In some embodiments, Compound 1 freebase is mixed with 3 ppm or less 4-fluoroaniline.

In some embodiments, Compound 1 hemifumarate is mixed with 3 ppm or less 4-fluoroaniline.

In some embodiments, Compound 1 freebase is mixed with 2 ppm or less 4-fluoroaniline.

In some embodiments, Compound 1 hemifumarate is mixed with 2 ppm or less 4-fluoroaniline.

In some embodiments, Compound 1 freebase is mixed with 1 ppm or less 4-fluoroaniline.

In some embodiments, Compound 1 hemifumarate is mixed with 1 ppm or less 4-fluoroaniline.

In some embodiments, the contaminants or contaminants or degradants comprise Compound a.

In some embodiments, the genotoxic contaminants or degradants comprise Compound a.

In some embodiments, Compound 1 freebase or Compound 1 hemifumarate is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less Compound a.

In some embodiments, Compound 1 freebase or Compound 1 hemifumarate is mixed with 75 ppm or less, 50 ppm or less, 25 ppm or less, 20 ppm or less, 15 ppm or less, 13 ppm or less, 10 ppm or less, 5 ppm or less, or 2.5 ppm or less Compound a.

In some embodiments, Compound 1 freebase is mixed with 15 ppm or less Compound a.

In some embodiments, Compound 1 hemifumarate is mixed with 15 ppm or less Compound a.

In some embodiments, Compound 1 freebase is mixed with 5 ppm or less Compound a.

In some embodiments, Compound 1 hemifumarate is mixed with 5 ppm or less Compound a.

In some embodiments, Compound 1 freebase is mixed with 4 ppm or less Compound a.

In some embodiments, Compound 1 hemifumarate is mixed with 4 ppm or less Compound a.

In some embodiments, Compound 1 freebase is mixed with 3 ppm or less Compound a.

In some embodiments, Compound 1 hemifumarate is mixed with 3 ppm or less Compound a.

In some embodiments, the contaminants or genotoxic contaminants or degradants comprise Compound b.

In some embodiments, Compound 1 freebase or Compound 1 hemifumarate is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less Compound b.

In some embodiments, Compound 1 freebase or Compound 1 hemifumarate is mixed with 75 ppm or less, 50 ppm or less, 25 ppm or less, 20 ppm or less, 15 ppm or less, 13 ppm or less, 10 ppm or less, 5 ppm or less, or 2.5 ppm or less Compound b.

In some embodiments, Compound 1 freebase is mixed with 75 ppm or less Compound b.

In some embodiments, Compound 1 hemifumarate is mixed with 75 ppm or less Compound b.

In some embodiments, Compound 1 freebase is mixed with 50 ppm or less Compound b.

In some embodiments, Compound 1 hemifumarate is mixed with 50 ppm or less Compound b.

In some embodiments, Compound 1 freebase is mixed with 15 ppm or less Compound b.

In some embodiments, Compound 1 hemifumarate is mixed with 15 ppm or less Compound b.

In some embodiments, Compound 1 freebase is mixed with 7 ppm or less Compound b.

In some embodiments, Compound 1 hemifumarate is mixed with 7 ppm or less Compound b.

In some embodiments, Compound 1 freebase or Compound 1 hemifumarate is mixed with at least two of 4-aminophenol, 4-fluoroaniline, Compound a, and Compound b, wherein the combined level of the at least two of 4-aminophenol, 4-fluoroaniline, Compounds a and b is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less.

In some embodiments, Compound 1 freebase or Compound 1 hemifumarate is mixed with Compound a and Compound b, wherein the combined level of Compound a and Compound b is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less. In some embodiments, the combined level of Compound a and Compound b is 100 ppm or less. In some embodiments, the combined level of Compound a and Compound b is 75 ppm or less. In some embodiments, the combined level of Compound a and Compound b is 50 ppm or less. In some embodiments, the combined level of Compound a and Compound b is 15 ppm or less.

In some embodiments, Compound 1 freebase or Compound 1 hemifumarate is mixed with at least three of 4-aminophenol, 4-fluoroaniline, Compound a, and Compound b, wherein the combined level of the at least three of 4-aminophenol, 4-fluoroaniline, Compounds a and b is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less.

In some embodiments, Compound 1 freebase or Compound 1 hemifumarate is mixed with 4-aminophenol, Compound a, and Compound b, wherein the combined level of 4-aminophenol, Compound a, and Compound b is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less. In some embodiments, the combined level of 4-aminophenol, Compound a, and Compound b is 100 ppm or less. In some embodiments, the combined level is 75 ppm or less. In some embodiments, the combined level is 50 ppm or less. In some embodiments, the combined level is 15 ppm or less.

In some embodiments, Compound 1 freebase or Compound 1 hemifumarate is mixed with 4-fluoroaniline, Compound a, and Compound b, wherein the combined level of 4-fluoroaniline, Compound a, and Compound b is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less. In some embodiments, the combined level of 4-fluoroaniline, Compound a, and Compound b is 100 ppm or less. In some embodiments, the combined level is 75 ppm or less. In some embodiments, the combined level is 50 ppm or less. In some embodiments, the combined level is 15 ppm or less.

In some embodiments, Compound 1 freebase or Compound 1 hemifumarate is mixed with 4-aminophenol, 4-fluoroaniline, Compound a, and Compound b, wherein the combined level of 4-aminophenol, 4-fluoroaniline, Compound a, and Compound b is 200 ppm or less, 100 ppm or less, 75 ppm or less, 50 ppm or less, 25 ppm or less, 20 ppm or less, 15 ppm or less, 13 ppm or less, 10 ppm or less, or 5 ppm or less. In some embodiments, the combined level of 4-aminophenol, 4-fluoroaniline, Compound a, and Compound b is 100 ppm or less. In some embodiments, the combined level is 75 ppm or less. In some embodiments, the combined level is 50 ppm or less. In some embodiments, the combined level is 15 ppm or less.

In some embodiments, the present disclosure provides a pharmaceutical composition comprising Compound 1 or Compound 1 hemifumarate, and one or more pharmaceutically acceptable excipients, wherein the pharmaceutical composition comprises 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less of contaminants or degradants comprising 4-aminophenol, 4-fluoroaniline, Compound a, Compound b, or mixtures thereof.

In some embodiments, the pharmaceutical composition is selected from any one of the compositions in the following table.

TABLE 1
Composition of Compound 1 Tablets, 20 mg,
40 mg, 60 mg, 80 mg, 100 mg and 120 mg

| | Composition | | | | | | |
|---|---|---|---|---|---|---|---|
| | mg/unit dose | | | | | | |
| | % | | | | | | |
| Ingredient | w/w | 20 mg | 40 mg | 60 mg | 80 mg | 100 mg | 120 mg |
| Compound 1 | 27.75 | 20¹ | 40² | 60³ | 80⁴ | 100⁵ | 120⁶ |
| Microcrystalline | 38.63 | 30.90 | 61.81 | 92.71 | 123.62 | 154.52 | 185.42 |
| Cellulose, PH-102 | | | | | | | |
| Lactose Anhydrous, | 19.32 | 15.46 | 30.91 | 46.37 | 61.82 | 77.28 | 92.74 |
| 60M | | | | | | | |
| Hydroxypropyl | 5.00 | 4.00 | 8.00 | 12.00 | 16.00 | 20.00 | 24.00 |
| Cellulose, EXF | | | | | | | |
| Croscarmellose | 6.00 | 4.80 | 9.60 | 14.40 | 19.20 | 24.00 | 28.80 |
| Sodium | | | | | | | |
| Colloidal Silicon | 0.30 | 0.24 | 0.48 | 0.72 | 0.96 | 1.20 | 1.44 |
| Dioxide | | | | | | | |
| Stearic Acid 50 | 3.00 | 2.40 | 4.80 | 7.20 | 9.60 | 12.00 | 14.40 |
| Total core | | 80.0 | 160.0 | 240.0 | 320.0 | 400.0 | 480.0 |
| tablet weight | | | | | | | |
| Opadry ® II | 4.00 | 3.20 | 6.40 | 9.60 | 12.80 | 16.00 | 19.20 |
| Blue (85F105057) | | | | | | | |
| Total coated | | 83.2 | 166.4 | 249.6 | 332.8 | 416.0 | 499.2 |
| tablet weight | | | | | | | |

¹20 mg of Compound 1 free base is equivalent to 22.20 mg of Compound 1 hemifumarate salt.
²40 mg of Compound 1 free base is equivalent to 44.40 mg of Compound 1 hemifumarate salt.
³60 mg of Compound 1 free base is equivalent to 66.60 mg of Compound 1 hemifumarate salt.
⁴80 mg of Compound 1 free base is equivalent to 88.80 mg of Compound 1 hemifumarate salt.
⁵100 mg of Compound 1 free base is equivalent to 111.00 mg of Compound 1 hemifumarate salt.
⁶120 mg of Compound 1 free base is equivalent to 132.20 mg of Compound 1 hemifumarate salt.

In one aspect, the present disclosure provides a compound of formula I.

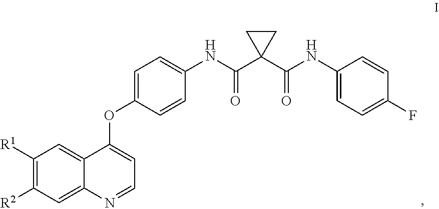

or a pharmaceutically acceptable salt thereof, comprising 200 ppm or less of contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl,
   wherein the compound of formula I is produced by a process comprising:
(b) reacting the compound of formula I-2 with Compound I-4 to yield the compound of formula I

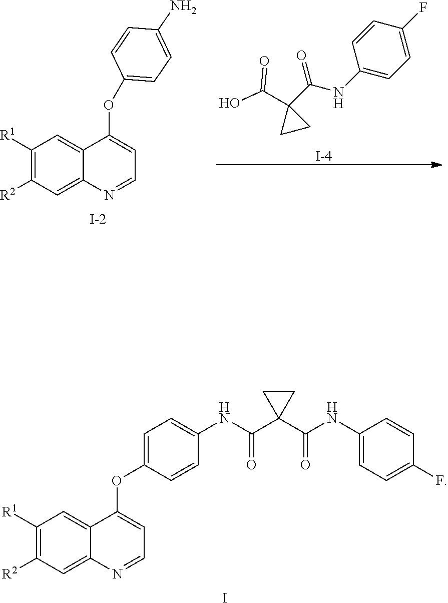

In one aspect, the present disclosure provides a compound of formula I:

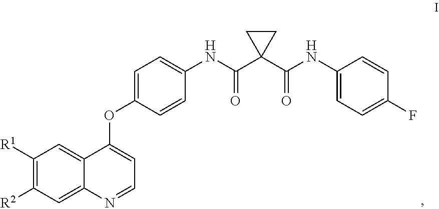

or a pharmaceutically acceptable salt thereof, comprising 100 ppm or less of genotoxic contaminants or degradants, wherein
R¹ is —C(O)—NH₂, —C(O)—NH(C₁₋₆ alkyl), or —C(O)—N(C₁₋₆ alkyl)₂; and
R² is —OC₁₋₆ alkyl,
   wherein the compound of formula I is produced by a process comprising:
(b) reacting the compound of formula I-2 with Compound I-4 to yield the compound of formula I

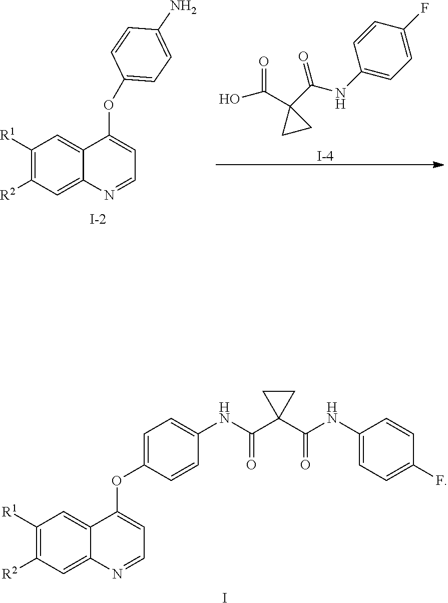

In one aspect, the present disclosure provides a compound of formula I:

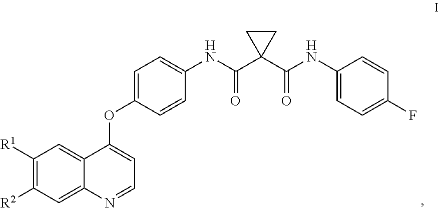

or a pharmaceutically acceptable salt thereof, comprising 200 ppm or less of contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl,
   wherein the compound of formula I is produced by a process comprising:
(a) reacting a compound of formula I-1′ with 4-aminophenol to yield a compound of formula I-2

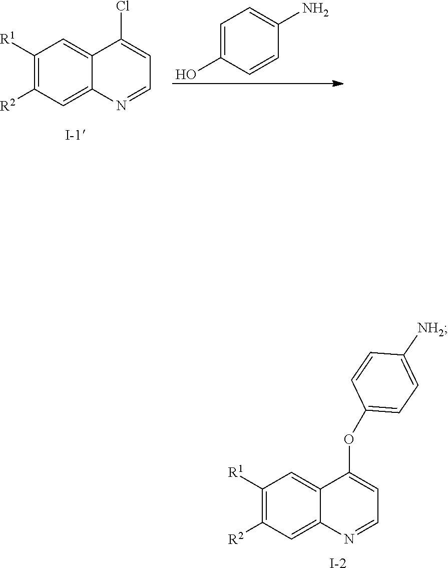

(b) reacting the compound of formula I-2 with Compound I-4 to yield the compound of formula I

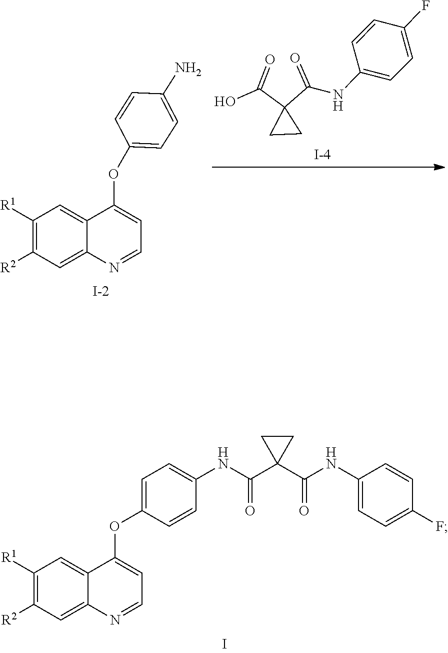

(c) optionally reacting compound of formula I with an acid.

In one aspect, the present disclosure provides a compound of formula I:

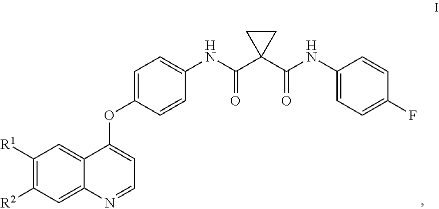

or a pharmaceutically acceptable salt thereof, comprising 100 ppm or less of genotoxic contaminants or degradants, wherein
R¹ is —C(O)—NH₂, —C(O)—NH(C₁₋₆ alkyl), or —C(O)—N(C₁₋₆ alkyl)₂; and
R² is —OC₁₋₆ alkyl,
   wherein the compound of formula I is produced by a process comprising:
(a) reacting a compound of formula I-1′ with 4-aminophenol to yield a compound of formula I-2

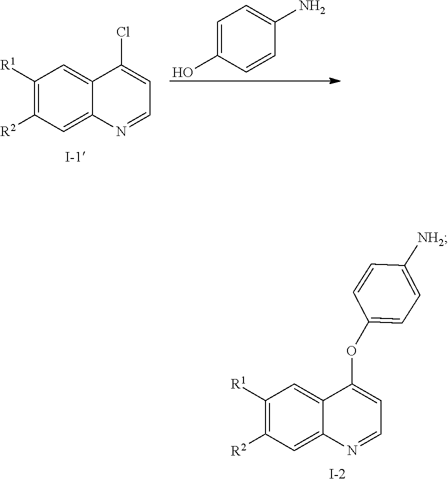

(b) reacting the compound of formula I-2 with Compound I-4 to yield the compound of formula I

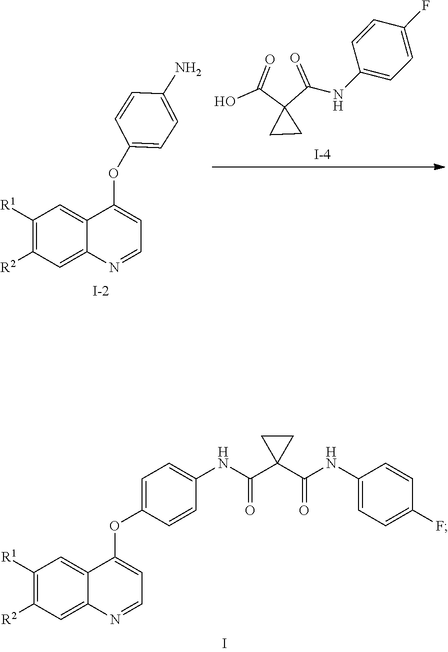

(c) optionally reacting compound of formula I with an acid.

In one aspect, the present disclosure provides a compound of formula I:

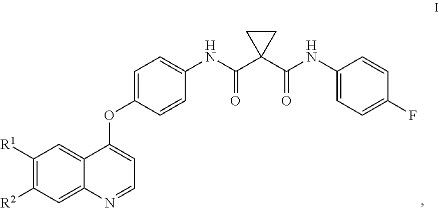

or a pharmaceutically acceptable salt thereof, comprising 200 ppm or less of contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl,
   wherein the compound of formula I is produced by a process comprising:
(b) reacting the compound of formula I-2 with Compound I-4 to yield the compound of formula I

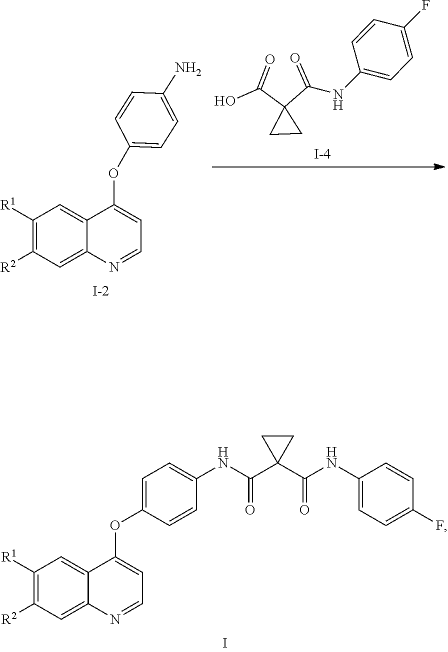

wherein the contaminants or degradants comprise 4-aminophenol, 4-fluoroaniline,

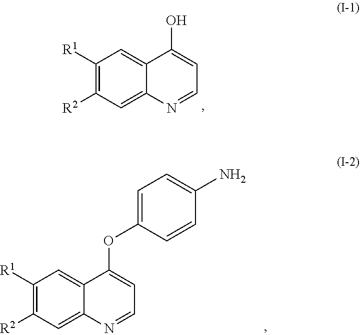

or mixtures thereof.

In one aspect, the present disclosure provides a compound of formula I:

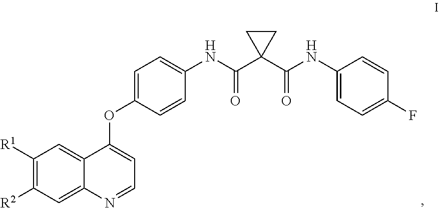

or a pharmaceutically acceptable salt thereof, comprising 100 ppm or less of genotoxic contaminants or degradants, wherein
R¹ is —C(O)—NH₂, —C(O)—NH(C₁₋₆ alkyl), or —C(O)—N(C₁₋₆ alkyl)₂; and
R² is —OC₁₋₆ alkyl,
   wherein the compound of formula I is produced by a process comprising:
(b) reacting the compound of formula I-2 with Compound I-4 to yield the compound of formula I

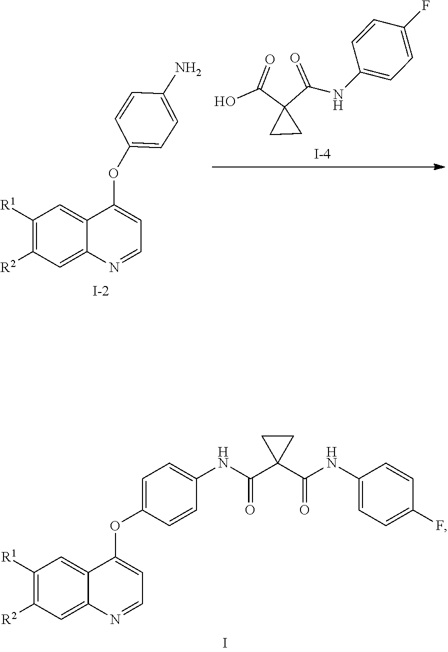

wherein the genotoxic contaminants or degradants comprise 4-aminophenol, 4-fluoroaniline,

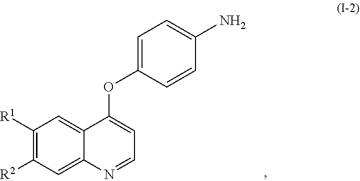

or mixtures thereof.

In one aspect, the present disclosure provides a compound of formula I:

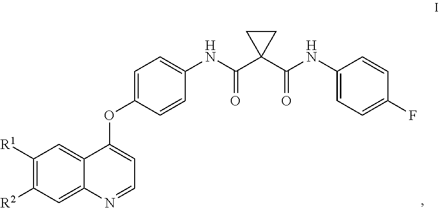

or a pharmaceutically acceptable salt thereof, comprising 200 ppm or less of contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl,
   wherein the compound of formula I or the salt is produced by a process comprising:
(a) reacting a compound of formula I-1′ with 4-aminophenol to yield a compound of formula I-2

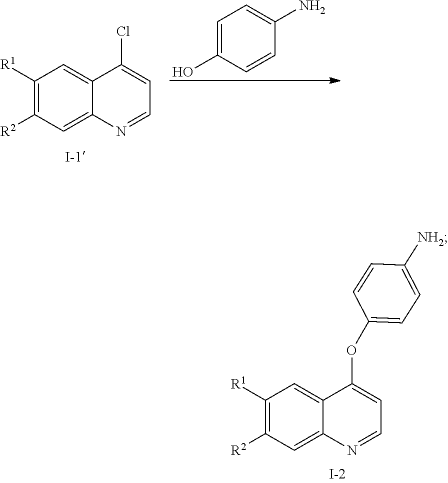

(b) reacting the compound of formula I-2 with Compound I-4 to yield the compound of formula I

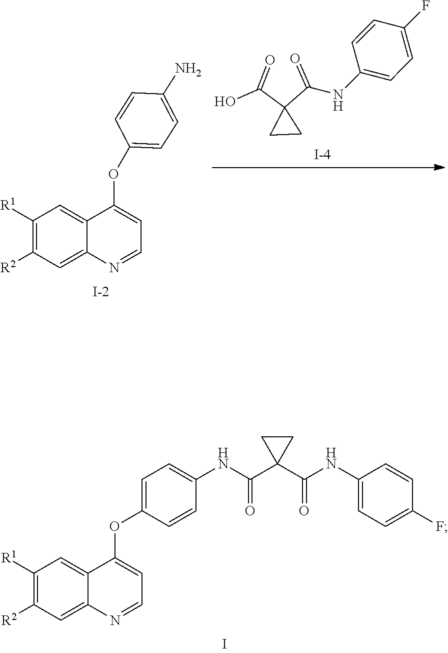

and
(c) optionally reacting compound of formula I with an acid,
   wherein the contaminants or degradants comprise 4-aminophenol, 4-fluoroaniline,

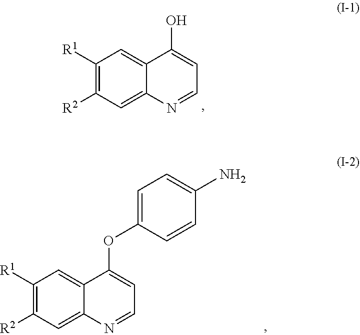

or mixtures thereof.

In one aspect, the present disclosure provides a compound of formula I.

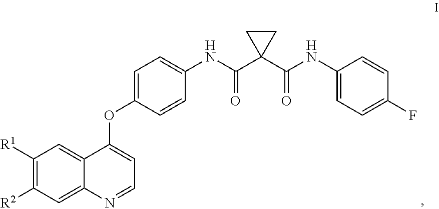

or a pharmaceutically acceptable salt thereof, comprising 100 ppm or less of genotoxic contaminants or degradants, wherein
R¹ is —C(O)—NH₂, —C(O)—NH(C₁₋₆ alkyl), or —C(O)—N(C₁₋₆ alkyl)₂; and
R² is —OC₁₋₆ alkyl,
   wherein the compound of formula I or the salt is produced by a process comprising:
(a) reacting a compound of formula I-1′ with 4-aminophenol to yield a compound of formula I-2

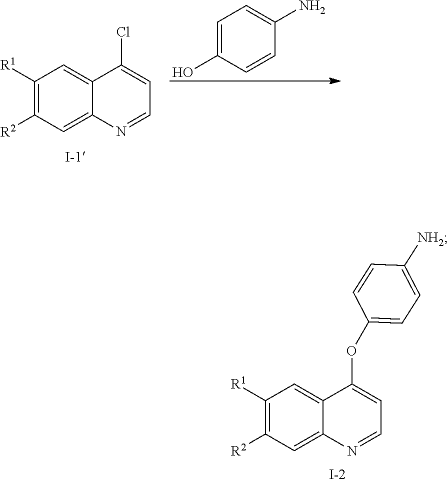

(b) reacting the compound of formula I-2 with Compound I-4 to yield the compound of formula I

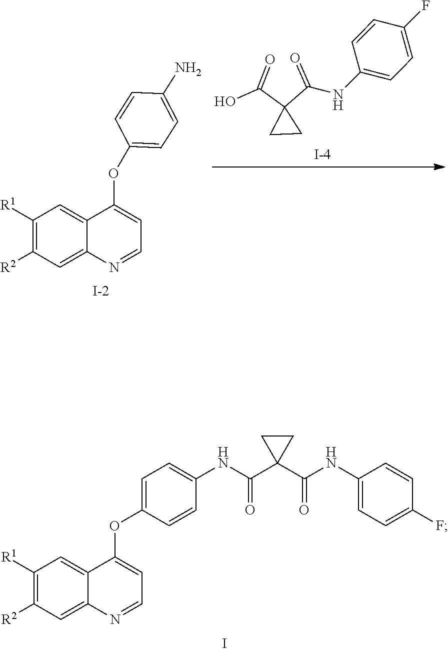

and
(c) optionally reacting compound of formula I with an acid,
   wherein the genotoxic contaminants or degradants comprise 4-aminophenol, 4-fluoroaniline,

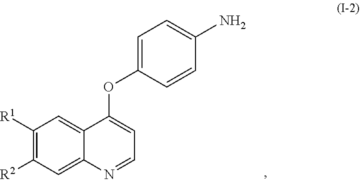

or mixtures thereof.

In one aspect, the present disclosure provides a process for making a compound of formula I:

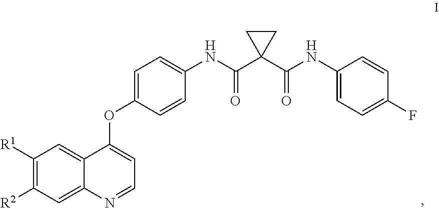

or a pharmaceutically acceptable salt thereof, wherein the compound of formula I or the salt comprises 200 ppm or less of contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl,
   wherein the process comprises:
(b) reacting the compound of formula I-2 with Compound I-4 to yield the compound of formula I

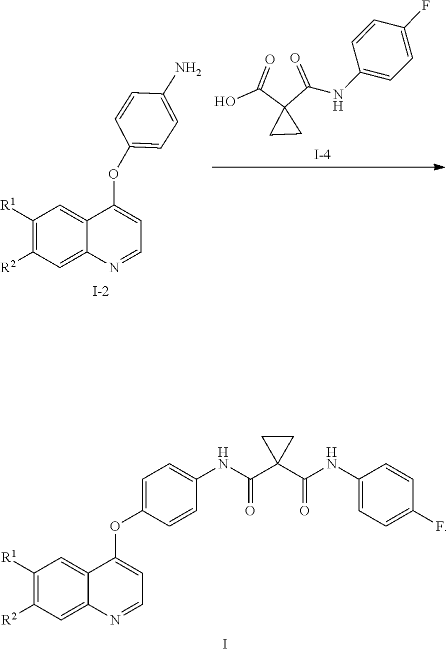

In one aspect, the present disclosure provides a process for making a compound of formula I:

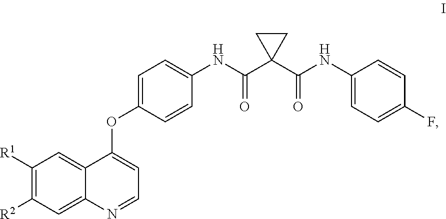

or a pharmaceutically acceptable salt thereof, wherein the compound of formula I or the salt comprises 100 ppm or less of contaminants or degradants, wherein
R¹ is —C(O)—NH₂, —C(O)—NH(C₁₋₆ alkyl), or —C(O)—N(C₁₋₆ alkyl)₂; and
R² is —OC₁₋₆ alkyl,
   wherein the process comprises:
(b) reacting the compound of formula I-2 with Compound I-4 to yield the compound of formula I

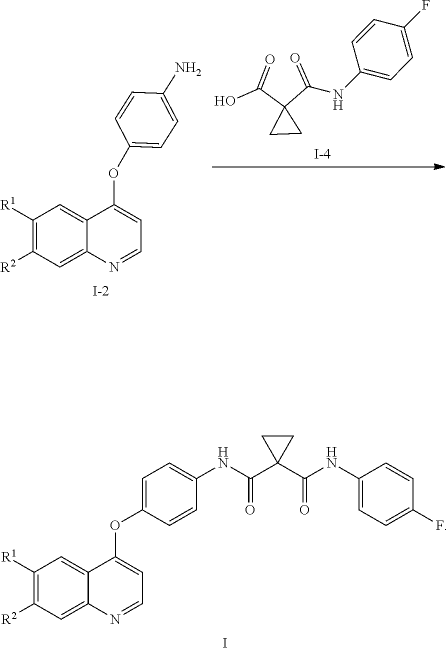

In one aspect, the present disclosure provides a process for making a compound of formula I:

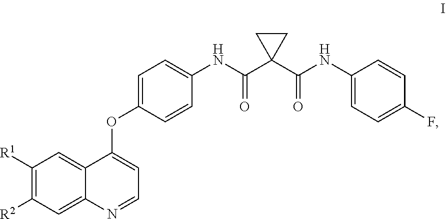

or a pharmaceutically acceptable salt thereof, wherein the compound of formula I or the salt comprises 200 ppm or less of contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl,
   wherein the process comprises:
(a) reacting a compound of formula I-1′ with 4-aminophenol to yield a compound of formula I-2

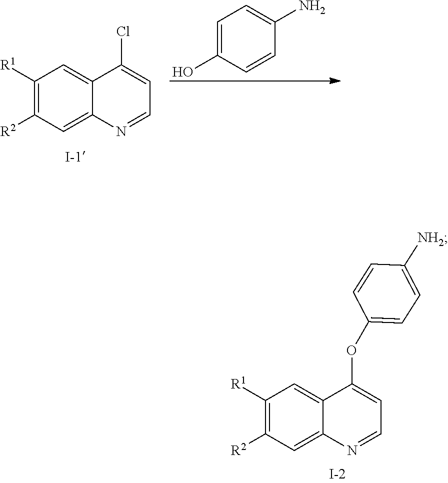

(b) reacting the compound of formula I-2 with Compound I-4 to yield the compound of formula I

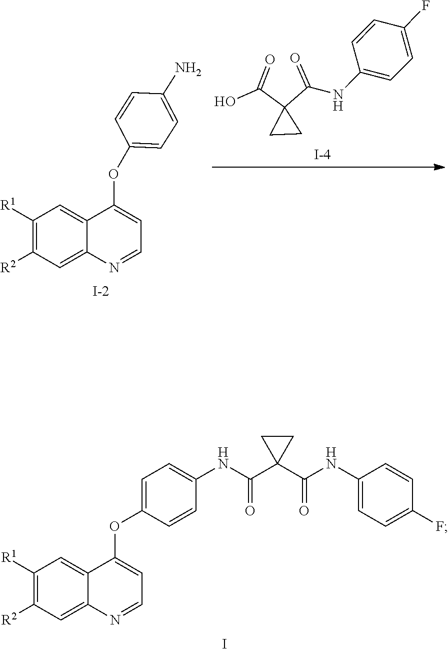

(c) optionally reacting compound of formula I with an acid,

In one aspect, the present disclosure provides a process for making a compound of formula I:

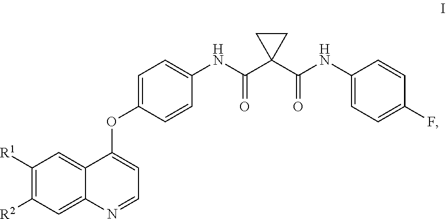

or a pharmaceutically acceptable salt thereof, wherein the compound of formula I or the salt comprises 100 ppm or less of contaminants or degradants, wherein
R¹ is —C(O)—NH₂, —C(O)—NH(C₁₋₆ alkyl), or —C(O)—N(C₁₋₆ alkyl)₂; and
R² is —OC₁₋₆ alkyl,
   wherein the process comprises:
(a) reacting a compound of formula I-1′ with 4-aminophenol to yield a compound of formula I-2

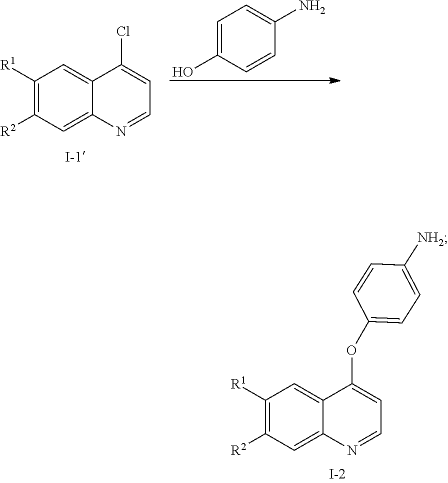

(b) reacting the compound of formula I-2 with Compound I-4 to yield the compound of formula I

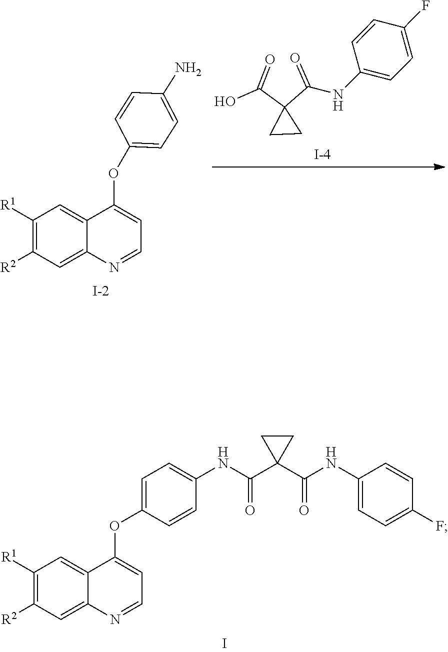

(c) optionally reacting compound of formula I with an acid.

In some embodiments, the contaminants or degradants comprise 4-aminophenol, 4-fluoroaniline,

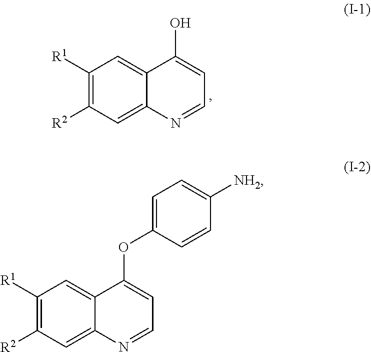

or mixtures thereof.

In some embodiments, the genotoxic contaminants or degradants comprise 4-aminophenol, 4-fluoroaniline,

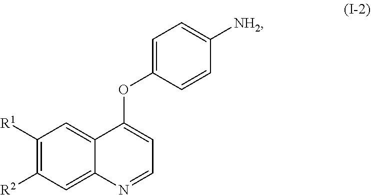

or mixtures thereof.

In some embodiments, the present disclosure provides a process for making a compound of formula I.

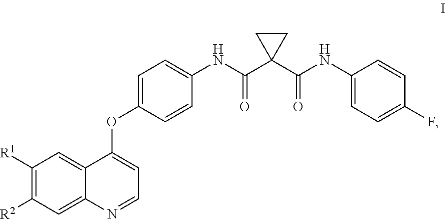

or a pharmaceutically acceptable salt thereof, wherein the compound of formula I or the salt comprises 200 ppm or less contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl,
   wherein the process comprises:
(b) reacting the compound of formula I-2 with Compound I-4 to yield the compound of formula I

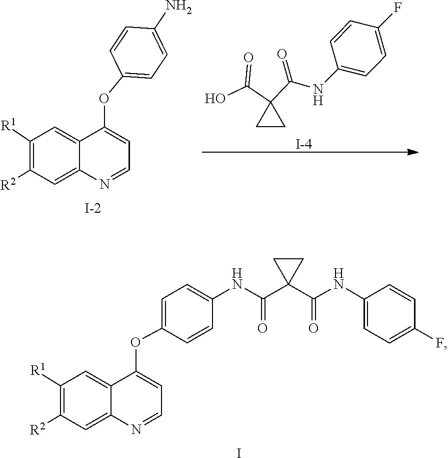

wherein the genotoxic contaminants or degradants comprise 4-aminophenol, 4-fluoroaniline,

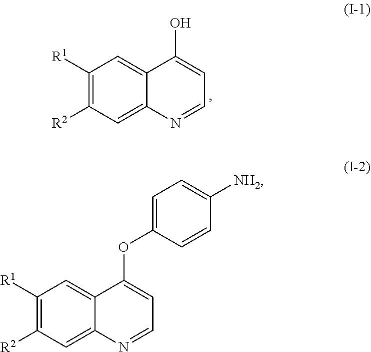

or mixtures thereof.

In some embodiments, the present disclosure provides a process for making a compound of formula I:

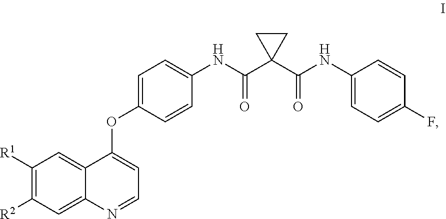

or a pharmaceutically acceptable salt thereof, wherein the compound of formula I or the salt comprises 200 ppm or less contaminants or degradants, wherein
R¹ is —COOH, —COO(C₁₋₆ alkyl), —C(O)—NH₂, —C(O)—NH(C₁₋₆ alkyl), or —C(O)—N(C₁₋₆ alkyl)₂; and
R² is —OC₁₋₆ alkyl,
   wherein the process comprises:
(a) reacting a compound of formula I-1′ with 4-aminophenol to yield a compound of formula I-2

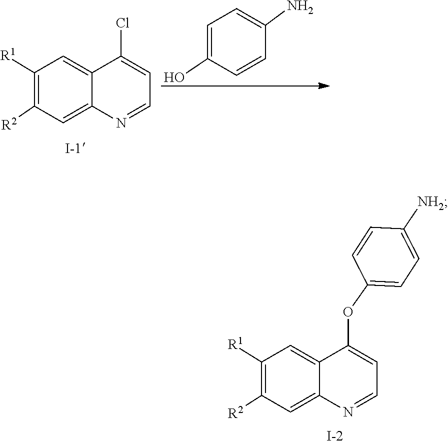

(b) reacting the compound of formula I-2 with Compound I-4 to yield the compound of formula I

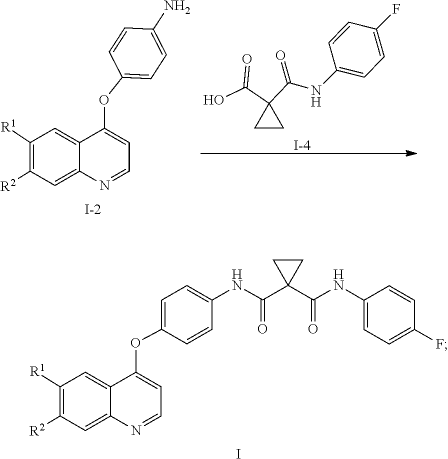

(c) optionally reacting compound of formula I with an acid,
   wherein the genotoxic contaminants or degradants comprise 4-aminophenol, 4-fluoroaniline,

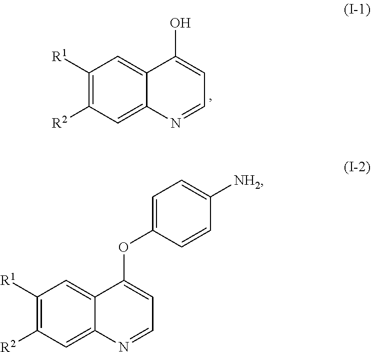

or mixtures thereof.

In some embodiments, the present disclosure provides a process for making a compound of formula I:

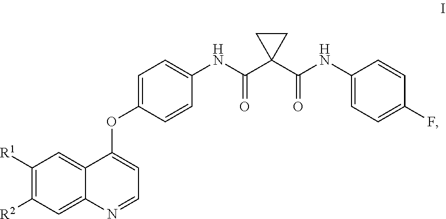

or a pharmaceutically acceptable salt thereof, wherein the compound of formula I or the salt comprises 100 ppm or less of genotoxic contaminants or degradants, wherein
R¹ is —C(O)—NH₂, —C(O)—NH(C₁₋₆ alkyl), or —C(O)—N(C₁₋₆ alkyl)₂; and
R² is —OC₁₋₆ alkyl,
   wherein the process comprises:
(b) reacting the compound of formula I-2 with Compound I-4 to yield the compound of formula I

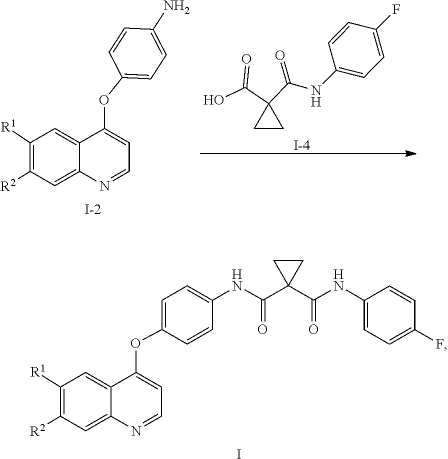

wherein the genotoxic contaminants or degradants comprise 4-aminophenol, 4-fluoroaniline,

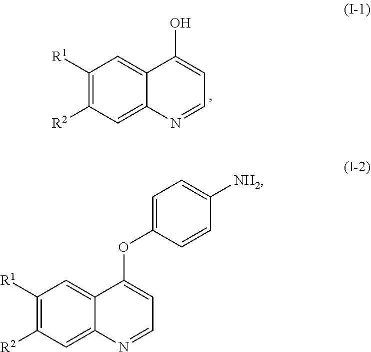

or mixtures thereof.

In some embodiments, the compound of formula I or the pharmaceutically acceptable salt thereof is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less impurities, contaminants, or degradants comprising 4-aminophenol, 4-fluoroaniline, (I-1), (I-2), or mixtures thereof.

In some embodiments, the compound of formula I or the pharmaceutically acceptable salt is mixed with 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less 4-aminophenol, 4-fluoroaniline, compound I-1, compound I-2, or mixtures thereof.

In some embodiments, the compound of formula I or the pharmaceutically acceptable salt thereof is mixed with 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less 4-aminophenol.

In some embodiments, the compound of formula I or the pharmaceutically acceptable salt thereof is mixed with 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less, or 1 ppm or less 4-fluoroaniline.

In some embodiments, the compound of formula I or the pharmaceutically acceptable salt thereof is mixed with 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less the compound of formula I-1.

In some embodiments, the compound of formula I or the pharmaceutically acceptable salt thereof is mixed with 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less the compound of formula I-2.

In some embodiments, the compound of formula I or the pharmaceutically acceptable salt thereof is mixed with 4-fluoroaniline and the compound of formula I-2, and the combined level of 4-fluoroaniline and the compound of formula I-2 is 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less.

In some embodiments, the compound of formula I or the pharmaceutically acceptable salt thereof is mixed with at least two of 4-aminophenol, 4-fluoroaniline, (I-1), and (I-2), wherein the combined level of the at least two of 4-aminophenol, 4-fluoroaniline, (I-1), and (I-2) is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less.

In some embodiments, the compound of formula I or the pharmaceutically acceptable salt thereof is mixed with at least three of 4-aminophenol, 4-fluoroaniline, (I-1), and (I-2), wherein the combined level of the at least three of 4-aminophenol, 4-fluoroaniline, (I-1), and (I-2) is 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, or 2.5 ppm or less.

In some embodiments, the compound of formula I or the pharmaceutically acceptable salt thereof is mixed 4-aminophenol, 4-fluoroaniline, (I-1), and (I-2), wherein the combined level of the at least four of 4-aminophenol, 4-fluoroaniline, (I-1), and (I-2) is 200 ppm or less, 100 ppm or less, 75 ppm or less, 50 ppm or less, 25 ppm or less, 20 ppm or less, 15 ppm or less, 13 ppm or less, 10 ppm or less, or 5 ppm or less.

In some embodiments, R¹ is —C(O)—NHMe and R² is —OMe.

In some embodiments, step (a) is carried out in the presence of sodium tert-butoxide or sodium tert-pentoxide and N,N-dimethylacetamide (DMA) at a temperature ranging from about 100° C. to about 130° C. In some embodiments, the temperature ranges from about 100° C. to about 120° C. In some embodiments, the temperature ranges from about 110° C. to about 120° C. In some embodiments, the temperature ranges from about 120° C. to about 130° C.

In some embodiments, step (a) is carried out in the presence of sodium tert-butoxide or sodium tert-pentoxide and DMA at a temperature ranging from about 60° C. to about 90° C. In some embodiments, the temperature ranges from about 60° C. to about 80° C. In some embodiments, the temperature ranges from about 70° C. to about 80° C. In some embodiments, the temperature ranges from about 70° C. to about 90° C.

In some embodiments, step (a) is carried out in the presence of sodium tert-butoxide or sodium tert-pentoxide and DMA at a temperature ranging from about 40° C. to about 90° C. In some embodiments, the temperature ranges from about 50° C. to about 70° C. In some embodiments, the temperature ranges from about 50° C. to about 60° C.

In some embodiments, the compound of formula I-2 is isolated by the following steps:
cooling the reaction;
charging water to the reaction to result in solids;
filtering the solids;
washing the solids with water and DMA; and drying the solids.

In some embodiments, step (b) comprises reacting Compound I-4 with a chlorinating agent to yield 1-(4-fluoro-phenylcarbamoyl)-cyclopropanecarbonyl chloride.

In some embodiments, the chlorinating agent is added to Compound I-4 in not less than 90 minutes.

In some embodiments, step (b) further comprises reacting the compound of formula I-2 with 1-(4-fluoro-phenylcarbamoyl)-cyclopropanecarbonyl chloride at a temperature ranging from about 10° C. to about 30° C. In some embodiments, the temperature ranges from about 10° C. to about 20° C. In some embodiments, the temperature ranges from about 10° C. to about 15° C. In some embodiments, the temperature ranges from about 5° C. to about 15° C. In some embodiments, the temperature ranges from about 20° C. to about 30° C. In some embodiments, the temperature ranges from about 25° C. to about 30° C.

In some embodiments, the compound of formula I is purified by the following steps: adding water to the reaction mixture;
stirring the reaction mixture at about 15-25° C. to result in solids;
filtering the solids;
washing the solids with water and tetrahydrofuran; and
drying the solids.

In some embodiments, the compound of formula I is purified by crystallization comprising:
cooling the reaction to about 15-25° C.;
charging with water over not less than 2 hours;
aging the reaction mixture for not less than 2 hour at about 15-25° C. to result a crystal slurry;
filtering the crystal slurry;
washing the crystal with water and tetrahydrofuran;
drying the crystals at non more than 40° C. jacket temperature; and recrystallizing compound I in tetrahydrofuran and 95:5 (v/v) water:ethanol.

In some embodiments, the compound of formula I is purified by the following steps:
adding water over not less than 1 hours;
stirring the reaction mixture for not less than 2 hour at about 15-25° C. to result in solids;
filtering the solids;
washing the solids with water and tetrahydrofuran; and
drying the solids.

In some embodiments, the compound of formula I is purified by the steps comprising:
adding water to the organic phase of the reaction mixture after phase separation at about 55-60° C. to result in a second mixture;
seeding the second mixture with Compound 1;
adding water to the seeded second mixture over at least one hour to result in a slurry;
cool the slurry to about 20-25° C. and aging the slurry;
filtering the slurry to yield solids;
washing the solids to yield Compound 1.

In some embodiments, step (c) comprises reacting the compound of formula I with fumaric acid in ethanol and water to produce the compound of formula I fumarate.

In some embodiments, the pharmaceutically acceptable salt is Compound 1 hemifumarate.

In one aspect, the present disclosure provides Compound 1:

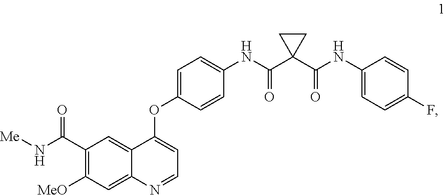 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, comprising 200 ppm or less of contaminants or degradants, wherein Compound 1 or the salt is produced by a process comprising:
(b) reacting Compound b with Compound I-4 to yield Compound 1

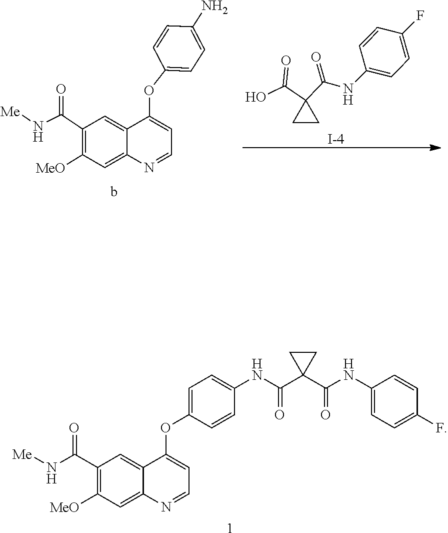

In one aspect, the present disclosure provides Compound 1:

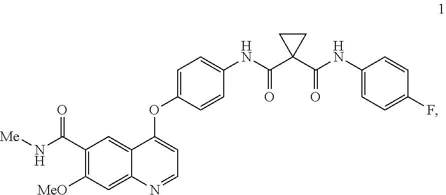 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, comprising 100 ppm or less of genotoxic contaminants or degradants, wherein Compound 1 or the salt is produced by a process comprising:
(b) reacting Compound b with Compound I-4 to yield Compound 1

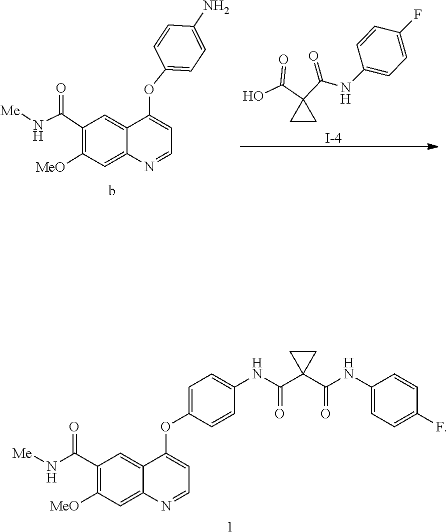

In one aspect, the present disclosure provides Compound 1:

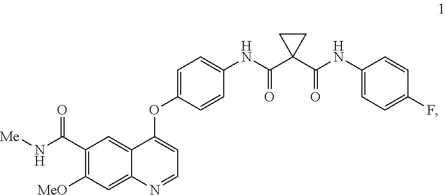 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, comprising 200 ppm or less of contaminants or degradants, wherein Compound 1 or the salt is produced by a process comprising:
(a) reacting Compound b′ with 4-aminophenol to yield Compound b

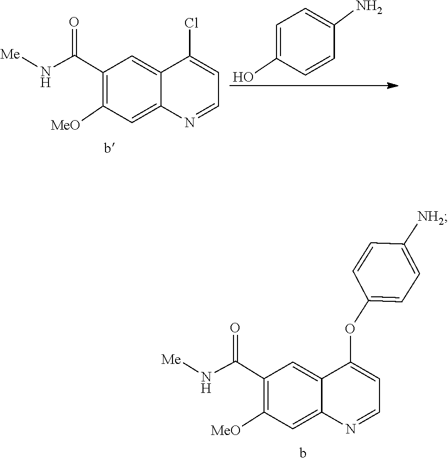

(b) reacting Compound b with Compound I-4 to yield Compound 1

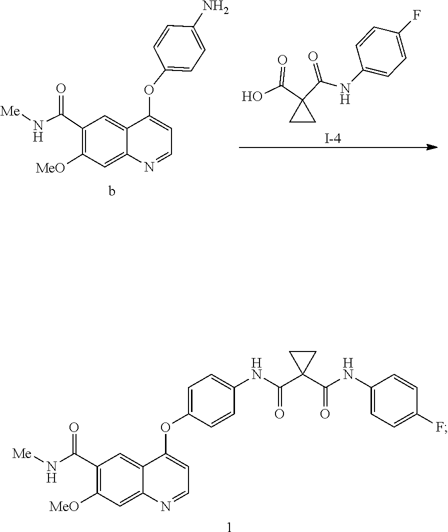

(c) optionally reacting Compound 1 with an acid.

In one aspect, the present disclosure provides Compound 1:

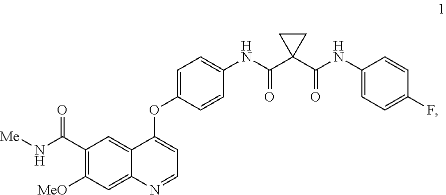 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, comprising 100 ppm or less of genotoxic contaminants or degradants, wherein Compound 1 or the salt is produced by a process comprising:
(a) reacting Compound b′ with 4-aminophenol to yield Compound b

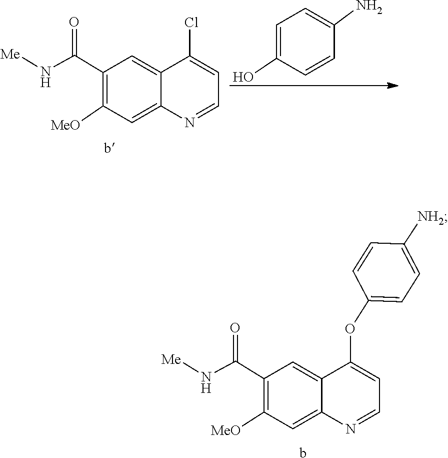

(b) reacting the Compound b with Compound I-4 to yield Compound 1

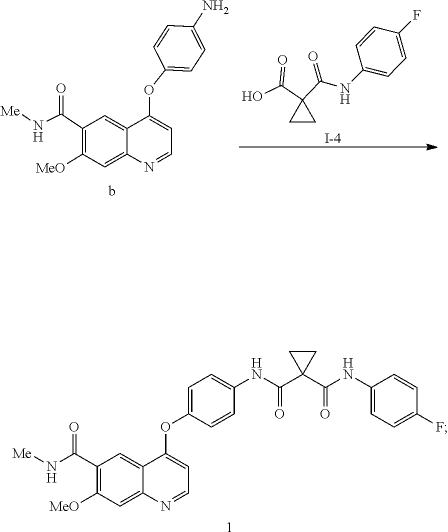

(c) optionally reacting Compound 1 with an acid.

In one aspect, the present disclosure provides a process for making Compound 1:

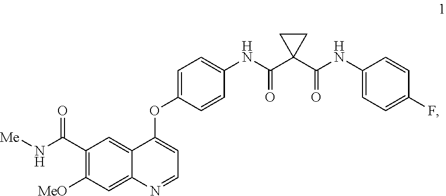 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, wherein Compound 1 or the salt comprises 200 ppm or less of contaminants or degradants, wherein the process comprises:
(b) reacting Compound b with Compound I-4 to yield Compound 1

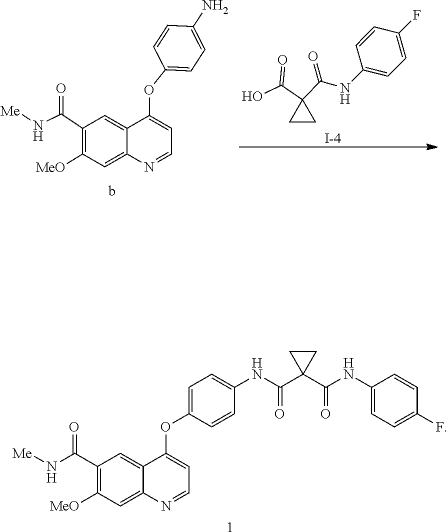

In one aspect, the present disclosure provides a process for making Compound 1:

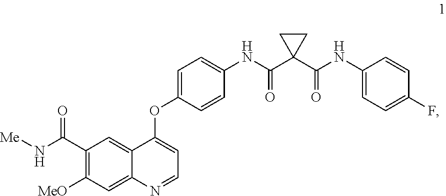 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, wherein Compound 1 or the salt comprises 100 ppm or less of genotoxic contaminants or degradants, wherein the process comprises:
(b) reacting Compound b with Compound I-4 to yield Compound 1

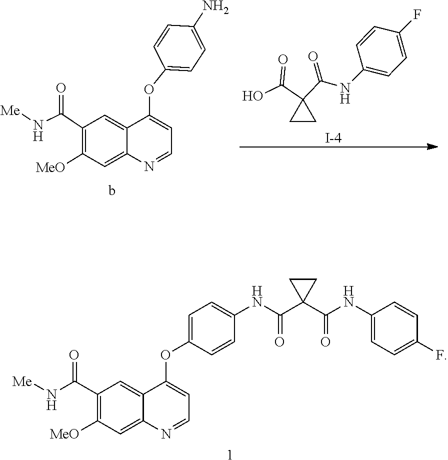

In one aspect, the present disclosure provides a process for making Compound 1:

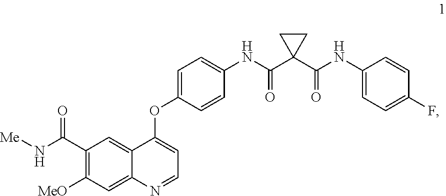 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, wherein Compound 1 or the salt comprises 200 ppm or less of contaminants or degradants, wherein the process comprises:
(a) reacting Compound b′ with 4-aminophenol to yield Compound b

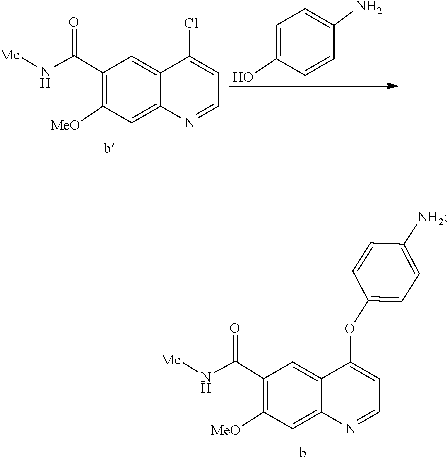

(b) reacting Compound b with Compound I-4 to yield Compound 1

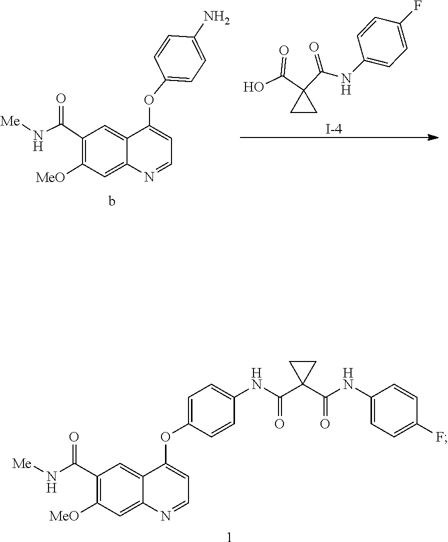

and
(c) optionally reacting Compound 1 with an acid.

In one aspect, the present disclosure provides a process for making Compound 1:

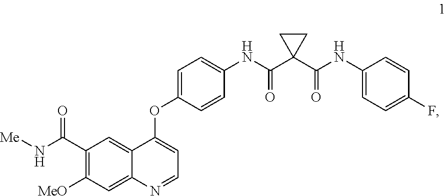 [SMILES: CNC(=O)c1cc2c(Oc3ccc(NC(=O)C4(C(=O)Nc5ccc(F)cc5)CC4)cc3)ccnc2cc1OC]

or a pharmaceutically acceptable salt thereof, wherein Compound 1 or the salt comprises 100 ppm or less of genotoxic contaminants or degradants, wherein the process comprises:
(a) reacting Compound b′ with 4-aminophenol to yield Compound b

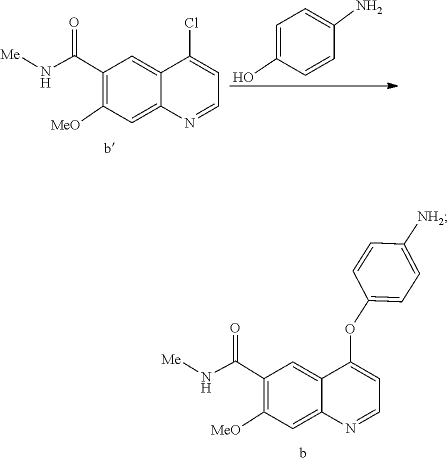

(b) reacting Compound b with Compound I-4 to yield Compound 1

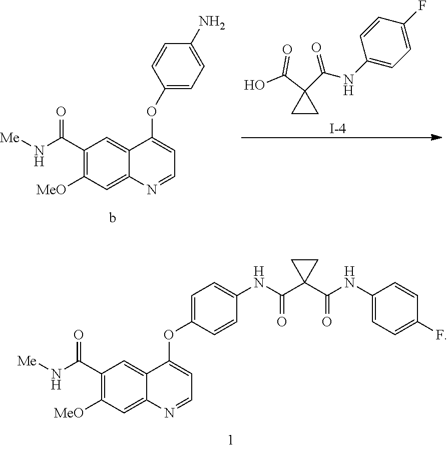

(c) optionally reacting Compound 1 with an acid.

In some embodiments, the contaminants or degradants comprise 4-aminophenol, 4-fluoroaniline,

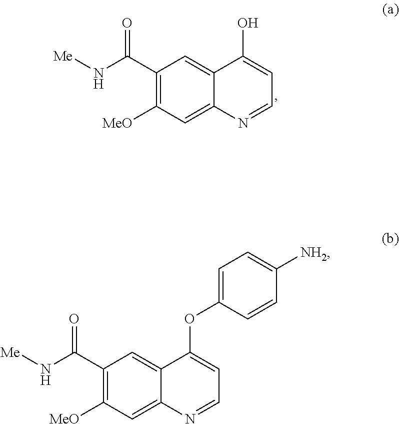 [SMILES: CNC(=O)c1cc2c(O)ccnc2cc1OC ; CNC(=O)c1cc2c(Oc3ccc(N)cc3)ccnc2cc1OC]

or mixtures thereof.

In some embodiments, the contaminants or degradants comprise genotoxic contaminants or degradants.

In some embodiments, the genotoxic contaminants or degradants comprise 4-aminophenol, 4-fluoroaniline,

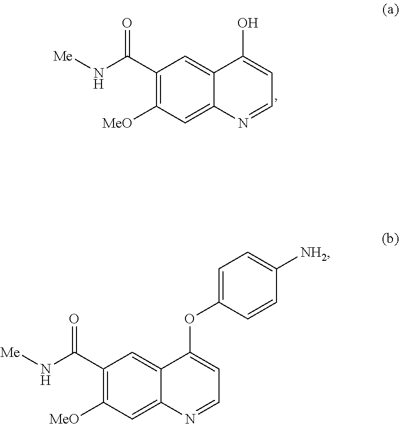 [SMILES: CNC(=O)c1cc2c(O)ccnc2cc1OC ; CNC(=O)c1cc2c(Oc3ccc(N)cc3)ccnc2cc1OC]

or mixtures thereof.

In some embodiments, the acid is fumaric acid.

In one aspect, the present disclosure provides Compound 1.

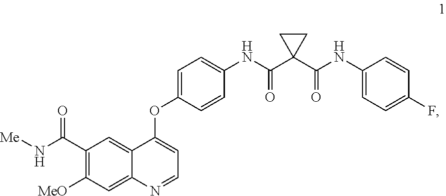 [SMILES: CNC(=O)c1cc2c(Oc3ccc(NC(=O)C4(C(=O)Nc5ccc(F)cc5)CC4)cc3)ccnc2cc1OC]

or Compound 1 fumaric acid salt, comprising 200 ppm or less of contaminants or degradants, wherein Compound 1 is produced by a process comprising:
(a) reacting Compound b′ with 4-aminophenol to yield Compound b

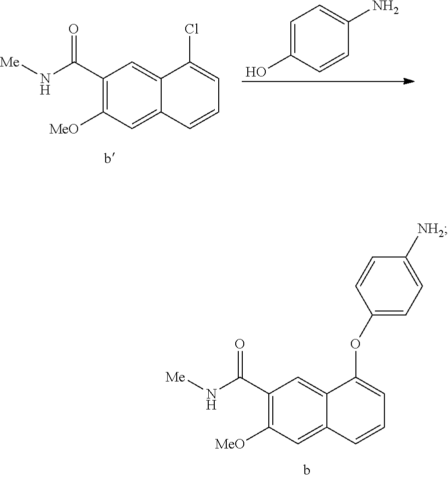

(b) reacting Compound b with Compound I-4 to yield Compound 1

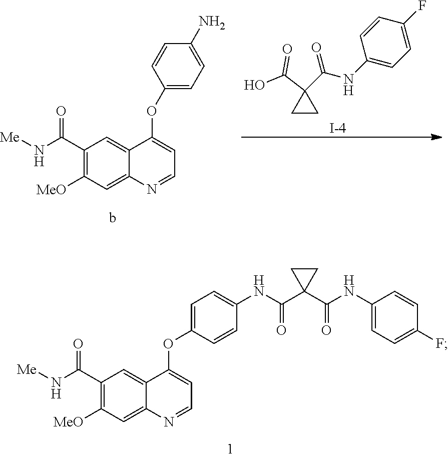

and
(c) optionally reacting Compound 1 with fumaric acid,
   wherein the contaminants or degradants comprise 4-aminophenol, 4-fluoroaniline,

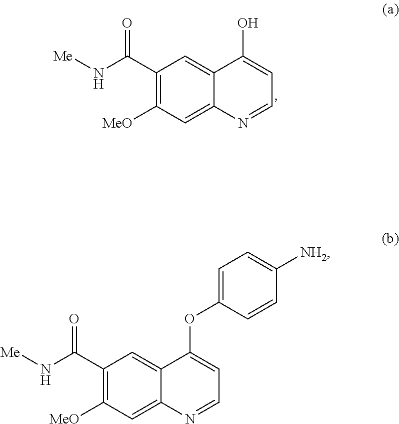 [SMILES: CNC(=O)c1cc2c(O)ccnc2cc1OC ; CNC(=O)c1cc2c(Oc3ccc(N)cc3)ccnc2cc1OC]

or mixtures thereof.

In one aspect, the present disclosure provides a process for making Compound 1 or Compound 1 fumaric acid salt, wherein Compound 1 or Compound 1 fumaric acid salt comprises 200 ppm or less of contaminants or degradants, wherein the process comprising:
(a) reacting Compound b′ with 4-aminophenol to yield Compound b

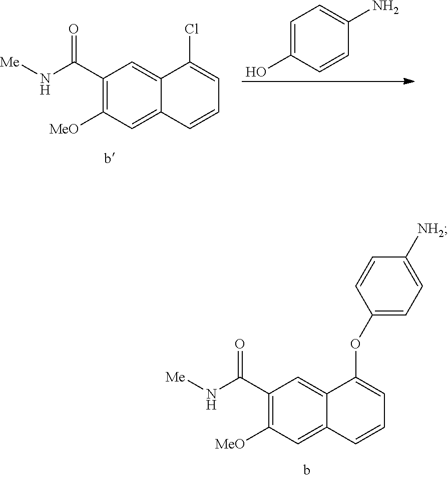

(b) reacting Compound b with Compound I-4 to yield Compound 1

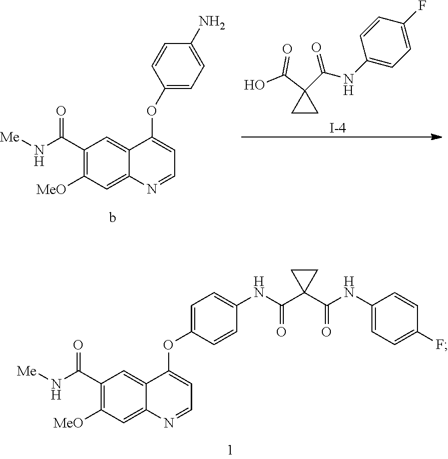

(c) optionally reacting Compound 1 with fumaric acid,
   wherein the contaminants or degradants comprise 4-aminophenol, 4-fluoroaniline,

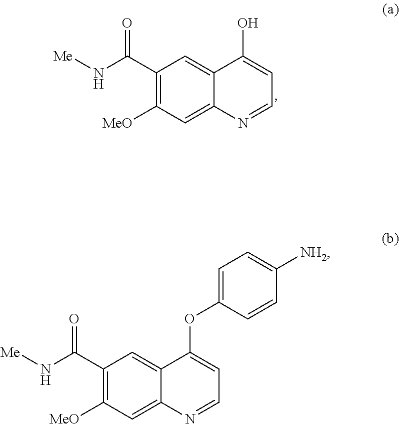 [SMILES: CNC(=O)c1cc2c(O)ccnc2cc1OC ; CNC(=O)c1cc2c(Oc3ccc(N)cc3)ccnc2cc1OC]

or mixtures thereof.

In some embodiments, the acid is fumaric acid and the pharmaceutically acceptable salt is Compound 1 hemifumarate.

In some embodiments, Compound 1 freebase or Compound 1 hemifumarate is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less impurities, contaminants, or degradants.

In some embodiments, Compound 1 freebase or Compound 1 hemifumarate is mixed with 200 ppm or less, 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less impurities, contaminants, or degradants comprising 4-aminophenol, 4-fluoroaniline, compounds a and b, or mixtures thereof.

In some embodiments, Compound 1 or its salt is mixed with 75 ppm or less, 50 ppm or less, 25 ppm or less, 20 ppm or less, 15 ppm or less, 13 ppm or less, 10 ppm or less, 5 ppm or less, or 2.5 ppm or less, or 1 ppm or less 4-aminophenol, 4-fluoroaniline, compound a, Compound b, or mixtures thereof.

In some embodiments, Compound 1 freebase is mixed with 15 ppm or less 4-aminophenol.

In some embodiments, Compound 1 hemifumarate is mixed with 15 ppm or less 4-aminophenol.

In some embodiments, Compound 1 freebase is mixed with 3 ppm or less 4-aminophenol.

In some embodiments, Compound 1 hemifumarate is mixed with 3 ppm or less 4-aminophenol.

In some embodiments, Compound 1 freebase is mixed with 15 ppm or less 4-fluoroaniline.

In some embodiments, Compound 1 hemifumarate is mixed with 15 ppm or less 4-fluoroaniline.

In some embodiments, Compound 1 freebase is mixed with 5 ppm or less, 3 ppm or less, 2 pm or less, or 1 ppm or less 4-fluoroaniline.

In some embodiments, Compound 1 hemifumarate is mixed with 5 ppm or less, 3 ppm or less, 2 pm or less, or 1 ppm or less 4-fluoroaniline.

In some embodiments, Compound 1 freebase is mixed with 15 ppm or less Compound a.

In some embodiments, Compound 1 hemifumarate is mixed with 15 ppm or less Compound a.

In some embodiments, Compound 1 freebase is mixed with 5 ppm or less, 4 ppm or less, or 3 pm or less Compound a.

In some embodiments, Compound 1 hemifumarate is mixed with 5 ppm or less, 4 ppm or less, or 3 pm or less Compound a.

In some embodiments, Compound 1 or Compound 1 hemifumarate is mixed with 75 ppm or less, 50 ppm or less, 15 ppm or less, or 10 pm or less Compound b.

In some embodiments, Compound 1 freebase is mixed with 75 ppm or less Compound b.

In some embodiments, Compound 1 hemifumarate is mixed with 75 ppm or less Compound b.

In some embodiments, Compound 1 freebase is mixed with 50 ppm or less Compound b.

In some embodiments, Compound 1 hemifumarate is mixed with 50 ppm or less Compound b.

In some embodiments, Compound 1 freebase is mixed with 15 ppm or less Compound b.

In some embodiments, Compound 1 hemifumarate is mixed with 15 ppm or less Compound b.

In some embodiments, Compound 1 freebase is mixed with 7 ppm or less Compound b.

In some embodiments, Compound 1 hemifumarate is mixed with 7 ppm or less Compound b.

In some embodiments, step (a) is carried out in the presence of sodium tert-butoxide or sodium tert-pentoxide and DMA at a temperature ranging from about 40° C. to about 90° C.

In some embodiments, step (a) is carried out in the presence of sodium tert-butoxide or sodium tert-pentoxide and DMA at a temperature ranging from about 60° C. to about 90° C.

In one embodiment, step (a) is performed at a temperature of 50-60° C.

In one embodiment, step (a) is performed at a temperature of 75-80° C.

In one embodiment, step (a) is performed at a temperature of 80-90° C.

In one embodiment, Compound b is isolated by adding water to the reaction mixture and isolating the solid product.

In some embodiments, Compound b is isolated by the following steps:
cooling the reaction to room temperature;
charging with water to the reaction to result in solids;
filtering the solids;
washing the solids with water and DMA; and
drying the solids at about 40-60° C. temperature.

In some embodiments, step (b) comprises reacting Compound I-4 with a chlorinating agent to yield 1-(4-fluoro-phenylcarbamoyl)-cyclopropanecarbonyl chloride (g). In one embodiment, the reaction is performed at a temperature that is 15° C. or less. In a further embodiment, the reaction is performed at a temperature that is 5-15° C. In a further embodiment, the reaction is performed at a temperature that is approximately 10-15° C. In a further embodiment, the reaction is performed at a temperature that is approximately 10-15° C. for 2-3 hours. In a further embodiment, the reaction is performed at room temperature for 2-4 hours. It was discovered that the generation of acid chloride (g) was fast and complete after approximately 15 minutes when carried out at room temperature. However, at room temperature, batch-to-batch inconsistency was observed, and some batches contained higher levels of impurities than others. It was further discovered that the production of unwanted side products was avoided by proceeding with the reaction at 10-15° C. At 10-15° C., the reaction was slower and generally took 2-3 hours to complete, but impurity levels could be controlled and minimized, especially that the level of 4-fluoroaniline in the final product was significantly reduced.

In another embodiment, the reaction with the chlorinating agent is performed in the presence of a catalytic amount of dimethylformamide.

In one embodiment, step (b) is performed in the presence of an organic solvent. In a further embodiment, the organic solvent is tetrahydrofuran.

In some embodiments, step (b) further comprises reacting Compound b with Compound I-4 at a temperature ranging from about 10° C. to about 30° C. In some embodiments, the temperature ranges from about 20° C. to about 25° C. In some embodiments, the temperature ranges from about 10° C. to about 15° C.

In one embodiment, Compound b is contacted with Compound g by adding a solution of compound g dissolved in a first solvent to a solution of Compound b dissolved in a second solvent, to create a reaction mixture.

In one embodiment, the first solvent is an organic solvent. In a further embodiment, the first solvent is a polar aprotic solvent. In a further embodiment, the first solvent is tetrahydrofuran.

In one embodiment, the second solvent is approximately 2:1 tetrahydrofuran:water by weight.

In one embodiment, compound g dissolved in a first solvent is added to a solution of Compound b dissolved in a second solvent over a period of approximately 30 minutes to approximately 1 hour. In another embodiment, compound g dissolved in a first solvent is added to a solution of Compound b dissolved in a second solvent over a period of no less than 30 minutes.

In one embodiment, the temperature of the reaction mixture of Compound g and Compound b is maintained at between approximately 20 to 27° C. In one embodiment, the reaction mixture is maintained at between approximately 25 to 27° C. In one embodiment, the reaction mixture is maintained at below approximately 27° C. In another embodiment, the reaction temperature is maintained at approximately 20 to 25° C. In another embodiment, the reaction temperature is maintained at approximately 10 to 15° C.

In another embodiment, the reaction mixture of Compound g and Compound b is heated to 35-40° C. and let stand to separate to an organic phase and an aqueous phase.

In another embodiment, the reaction mixture is heated to 35-45° C. and let stand to separate to an organic phase and an aqueous phase.

In one embodiment, the process further comprises discarding the aqueous phase, heating the organic phase to 45-50° C., and then filtering the organic phase at 45-50° C.

In one embodiment, the process further comprises discarding the aqueous phase, heating the organic phase to 55-60° C., and then filtering the organic phase at 55-60° C.

In one embodiment, the process further comprises discarding the aqueous phase, and heating the organic phase to 55-60° C.

In one embodiment, the process further comprises discarding the aqueous phase, heating the organic phase to 35-45° C.

In another embodiment, the process further comprises cooling the organic phase to 20-25° C. and adding water to the organic phase to create a second mixture, wherein the volume of water added is approximately 1.5 to approximately 2.5 times the volume of the organic phase.

In another embodiment, the process further comprises adding water to the organic phase to create a second mixture while maintaining a temperature of 50-55° C.

In another embodiment, the process further comprises adding water to the organic phase to create a second mixture while maintaining a temperature of 55-60° C.

In another embodiment, the process further comprises adding water to the organic phase to create a second mixture while maintaining a temperature of 35-45° C.

In one embodiment, the water is added to the organic phase over a period of at least one hour. In another embodiment, the water is added to the organic phase over a period of approximately 4 to 4.5 hours.

In one embodiment, the second mixture is stirred for at least 12 hours, and Compound 1 is a solid, which is collected by filtration or the like. In another embodiment, the second mixture is stirred for at least 2 hours, and the product is collected by filtration or the like. In one embodiment, the second mixture is stirred for at least 2 hours at a temperature of 35-45° C., and the crude product is collected by filtration or the like.

In one embodiment, the crude product is purified by polish filtration and recrystallization.

In some embodiments, Compound 1 is purified by the following steps:
adding water over not less than 1 hours;
stirring the reaction mixture for not less than 2 hour at about 15-25° C. to result in solids;
filtering the solids;
washing the solids with water and tetrahydrofuran; and
drying the solids.

In some embodiments, Compound 1 is purified by the following steps:
adding water to the organic phase of the reaction mixture after phase separation at about 55-60° C. to result in a second mixture;
seeding the second mixture with Compound 1;
adding water to the seeded second mixture over at least one hour to result in a slurry;
cool the slurry to about 20-25° C. and aging the slurry;
filtering the slurry to yield solids;
washing the solids with THE and water to yield Compound 1.

In some embodiments, step (c) comprises reacting Compound 1 with fumaric acid in ethanol and water to produce Compound 1 hemifumarate.

In one embodiment, step (c) is performed in the presence of a solvent. In a further embodiment, the solvent is selected from water, an alcoholic solvent, THF, DMF, MEK, acetonitrile, 1,4-dioxane, and MTBE, or any combination thereof. In a further embodiment, the solvent is a mixture of water in an alcoholic solvent.

In one embodiment, the alcoholic solvent is selected from methanol, ethanol, n-propanol, isopropanol, n-butanol, t-butanol, pentanol, hexanol, heptanol, and octanol.

In a further embodiment, the solvent is a 20% solution of water in ethanol.

In a further embodiment, the solvent is a 5% solution of water in ethanol.

In one embodiment, the volume of the 20% solution of water in ethanol used in the reaction is about 2-3 time the weight of Compound 1. In another embodiment, the volume (mL) of the 20% solution of water in ethanol used in the reaction is about 3 time the weight (gram) of Compound 1.

In one embodiment, the quantity of fumaric acid used is about 0.5-1.0 stoichiometric equivalents with respect to Compound 1. In another embodiment, the quantity of fumaric acid used is about 0.75-1.0 stoichiometric equivalents with respect to Compound 1. In another embodiment, the quantity of fumaric acid used is about 0.8-0.82 stoichiometric equivalents with respect to Compound 1.

In one embodiment, Compound 1 is reacted with fumaric acid by adding a mixture of fumaric acid dissolved in a 20% solution of water in ethanol at 45-50° C. to Compound 1 to create a reaction mixture.

In one embodiment, the volume (mL) of the 20% solution of water in ethanol used for dissolving fumaric acid is about 2-3 times the weight (gram) of Compound 1. In another embodiment, the volume of the 20% solution of water in ethanol used for dissolving fumaric acid is about 2.2-2.8 times the weight of Compound 1. In another embodiment, the volume of the 20% solution of water in ethanol used for dissolving fumaric acid is about 2.4-2.6 times the weight of Compound 1.

In one embodiment, step (c) further comprises heating the reaction mixture to reflux temperature and stirring. In another embodiment, the refluxing reaction mixture is stirred for 4-6 hours.

In one embodiment, step (c) further comprises cooling the reaction mixture and separating the solid product from the solvent.

### EXAMPLES

### Compound 1 Hemifumarate Preparative Example 1

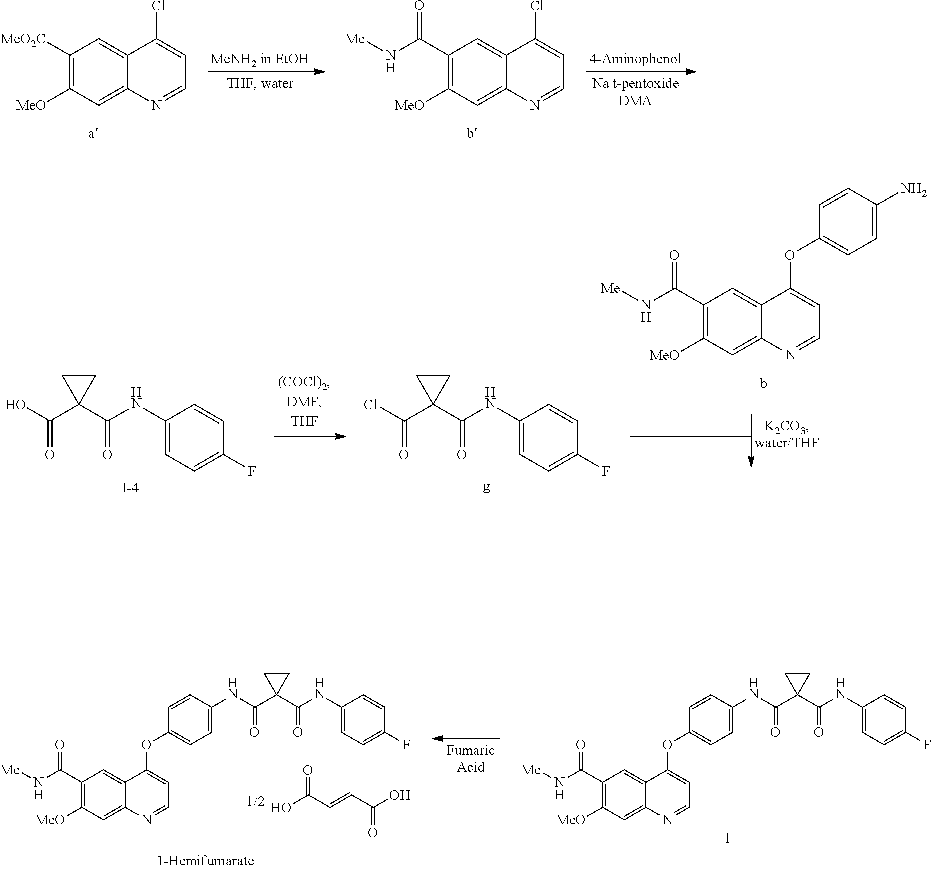

### Synthesis of 4-chloro-7-methoxy-N-methylquinoline-6-carboxamide

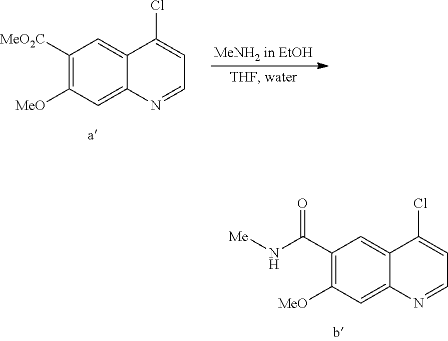 [SMILES: COC(=O)c1cc2c(Cl)ccnc2cc1OC ; CNC(=O)c1cc2c(Cl)ccnc2cc1OC]

To a suspension of methyl 4-chloro-7-methoxyquinoline-6-carboxylate a′ (2 g, 8 mmol) in THF (20 mL) was added methyl amine in EtOH (33% w/w, 8 M, 20 mL, 160 mmol) and H₂O (10 mL). The resulting mixture was stirred at room temperature. The mixture turned into a clear solution in about 10 min and remained as a clear solution during the reaction. The stirring was continued until the starting material was completely consumed as evidenced by LCMS and HPLC. It took about 3 hours. The mixture was then concentrated and the residue was slurried in 20 mL of water, and filtered using a filter funnel. Some EtOAc was used to transfer the material from the flask to the filter funnel. The product was dried to give 4-chloro-7-methoxy-N-methylquinoline-6-carboxamide as white solid (yield 1.8 g, 90%, HPLC purity >97%).

### Synthesis of 4-(4-aminophenoxy)-7-methoxy-N-methylquinoline-6-carboxamide

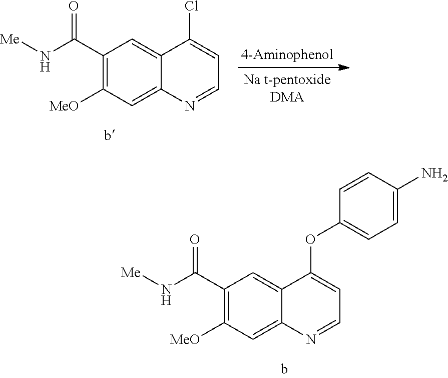 [SMILES: CNC(=O)c1cc2c(Cl)ccnc2cc1OC ; CNC(=O)c1cc2c(Oc3ccc(N)cc3)ccnc2cc1OC]

A 5 L, 3-neck round bottom flask equipped with a thermometer, nitrogen inlet, and magnetic stirrer was charged with 4-chloro-7-methoxy-N-methylquinoline-6-carboxamide (b′; 300 g; 1 eq.), 4-aminophenol (195.9 g; 1.5 eq.), and DMA (1500 mL). The resulting solution was stirred at room temperature, and a solution of sodium t-pentoxide (184.52 g; 1.4 eq.) dissolved in anhydrous THE (313 mL) was added with stirring over a 5 minute period. The reaction mixture was then heated to 75-80° C. and stirred for an additional 2-6 hours. The reaction mixture was then cooled to room temperature and charged with water (3 L), and stirred at least for an additional 1 hour. The product was filtered and washed twice with 600 mL of 1:1 DMA/water, then once with 1200 mL water. The product was transferred to a crystallizing dish and dried in the vacuum oven at 40-45° C. for a minimum of 18 hours to yield a light brown shiny solid (370-377 g; 96-97%).

### Synthesis of 1-((4-fluorophenyl)carbamoyl)cyclopropane-1-carbonyl chloride—Method 1

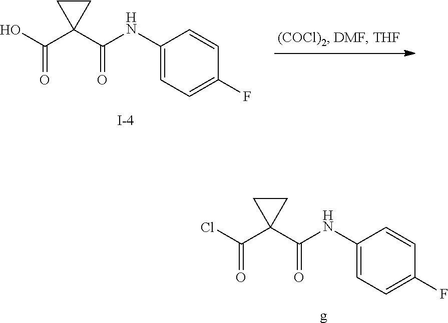

A 250 mL, 3 neck round bottom flask equipped with a thermometer, nitrogen inlet, and magnetic stirrer was charged with 1-((4-fluorophenyl)carbamoyl)cyclopropane-1-carboxylic acid (I-4, 19.11 g; 1.3 eq.), 75 mL anhydrous THF, and 0.25 mL DMF (catalyst). The mixture was stirred until all solids dissolved, cooled to 5-10° C., and then charged with oxalyl chloride (7.13 mL; 1.28 eq.). The resulting mixture was aged at 10-15° C. for 2-3 hours and reaction completion was confirmed by IPC (in process control). Upon reaction completion, the resulting product mixture was used in the next step without further purification.

### Synthesis of 1-((4-fluorophenyl)carbamoyl)cyclopropane-1-carbonyl chloride—Method 2

A 250 mL, 3 neck round bottom flask equipped with a thermometer, nitrogen inlet, and magnetic stirrer was charged with 1-((4-fluorophenyl)carbamoyl)cyclopropane-1-carboxylic acid (I-4, 19.11 g; 1.3 eq.), 75 mL anhydrous THF, and 0.25 mL DMF (catalyst). The mixture was stirred until all solids dissolved, cooled to 5-15° C., and then charged with oxalyl chloride (7.13 mL; 1.28 eq.). The resulting mixture was warmed to room temperature and then stirred for 2-4 hours. The resulting product mixture was used in the next step without further purification.

### Synthesis of N-(4-fluorophenyl)-N-(4-((7-methoxy-6-(methylcarbamoyl)quinolin-4-yl)oxy)phenyl)cyclopropane-1,1-dicarboxamide (1)—Method 1

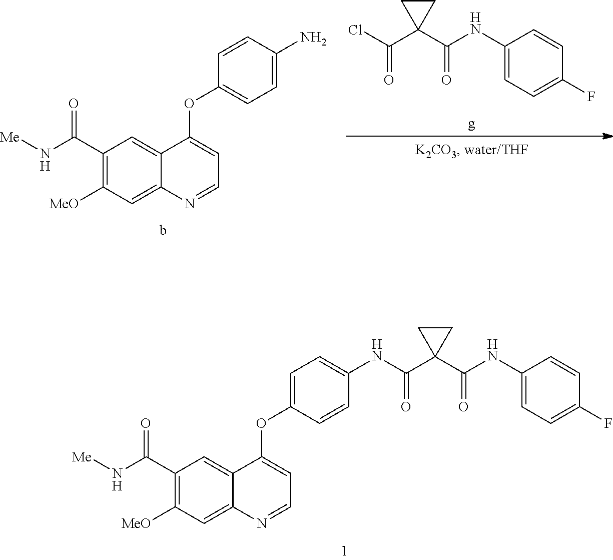 [SMILES: CNC(=O)c1cc2c(Oc3ccc(N)cc3)ccnc2cc1OC ; O=C(Cl)C1(C(=O)Nc2ccc(F)cc2)CC1 ; CNC(=O)c1cc2c(Oc3ccc(NC(=O)C4(C(=O)Nc5ccc(F)cc5)CC4)cc3)ccnc2cc1OC]

A 500 mL, 3 neck round bottom flask equipped with a thermometer, nitrogen inlet, and magnetic stirrer was charged with 4-(4-aminophenoxy)-7-methoxy-N-methylquinoline-6-carboxamide (b, 21.3 g; 1.0 eq.), 210 mL anhydrous THF, and a solution composed of potassium carbonate (27.32 g; 3 eq) and 100 mL water. The added aqueous K₂CO₃ solution was rinsed forward with an additional 6.4 mL water. With vigorous agitation, the reaction mixture containing Compound g from the previous example was transferred to the present reaction mixture over a period of no less than 30 minutes while maintaining an internal temperature between 2° and 25° C. The transfer equipment was rinsed with 32 mL of anhydrous THF. The reaction mixture was agitated at ambient temperature for 0.5-1 hour. The resulting mixture was warmed to 35-40° C. and the phases were allowed to separate. The lower aqueous layer was discarded and the top organic phase was warmed to 55-60° C. and then polish filtered and rinsed with 21 mL of THF. The filtered organic phase was transferred to a 1 L, 3 neck round bottom flask equipped with thermometer, nitrogen inlet, and mechanical stirring and charged, with water at 55-60° C. The resulting solution was seeded with Compound 1 and to the resulting seed bed water was added as an anti-solvent over 4-4.5 hours while maintaining a temperature of 50-55° C. The resulting slurry was cooled to 20-25° C. and aged for no less than 2 hours. The product was then filtered, washed with water/THF and dried.

### Synthesis of N-(4-fluorophenyl)-N-(4-((7-methoxy-6-(methylcarbamoyl)quinolin-4-yl)oxy)phenyl)cyclopropane-1,1-dicarboxamide (1)—Method 2

A 500 mL, 3 neck round bottom flask equipped with a thermometer, nitrogen inlet, and magnetic stirrer was charged with 4-(4-aminophenoxy)-7-methoxy-N-methylquinoline-6-carboxamide (b, 21.3 g; 1.0 eq.), 210 mL anhydrous THF, and a solution composed of potassium carbonate (27.32 g; 3 eq) and 100 mL water. The added aqueous K₂CO₃ solution was rinsed forward with an additional 6.4 mL water. With vigorous agitation, the reaction mixture containing Compound g from the previous example was transferred to the present reaction mixture over a period of 0.5-1 hour while maintaining an internal temperature below 27° C. The transfer equipment was rinsed with 32 mL of anhydrous THF. The reaction mixture was agitated at ambient temperature for 0.5-1 hour. The resulting mixture was warmed to 35-40° C. and the phases was allowed to separate. The lower aqueous layer was discarded and the top organic phase was warmed to 45-50° C. and then filtered through a filter paper and rinsed with 21 mL of THF. The filtered organic phase was transferred to a 1 L, 3 neck round bottom flask equipped with thermometer, nitrogen inlet, and mechanical stirring and charged, over a minimum of 1 hour with 694 mL of filtered water. The resulting mixture was stirred at 20-25° C. for a minimum of 12 hours, and the product was then filtered and rinsed twice with 42 mL of a 2:1 water:THF mixture. The product was then dried on a filter paper at room temperature or in a vacuum oven at 40-45° C. to yield a white to beige solid (31.36 g; 90%).

### Synthesis of N-(4-fluorophenyl)-N-(4-((7-methoxy-6-(methylcarbamoyl)quinolin-4-yl)oxy)phenyl)cyclopropane-1,1-dicarboxamide·1/2 fumaric acid (1 hemifumarate)—Method 1

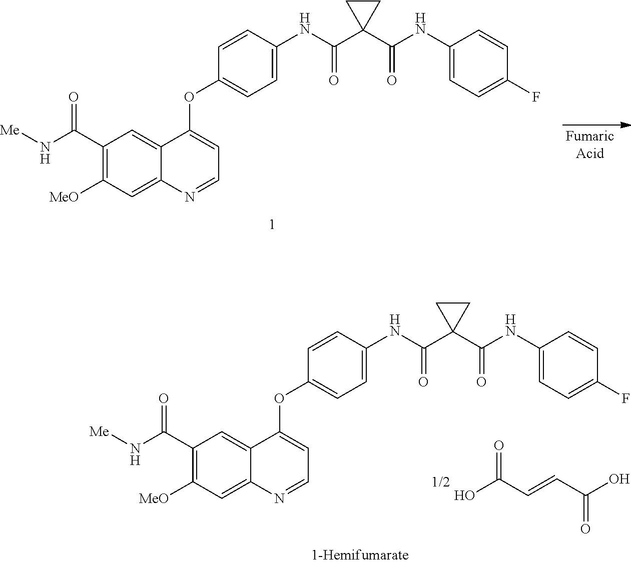 [SMILES: CNC(=O)c1cc2c(Oc3ccc(NC(=O)C4(C(=O)Nc5ccc(F)cc5)CC4)cc3)ccnc2cc1OC ; CNC(=O)c1cc2c(Oc3ccc(NC(=O)C4(C(=O)Nc5ccc(F)cc5)CC4)cc3)ccnc2cc1OC ; O=C(O)/C=C/C(=O)O]

A 2000 mL, 3 neck round bottom flask equipped with a thermometer, nitrogen inlet, and magnetic stirrer was charged with fumaric acid (80 g; 0.82 eq.) and 1.2 L of a 20% solution of water in ethanol. The mixture was heated to 45-50° C. and stirred until all solids were dissolved. To a separate 3 L, 3 neck round bottom flask equipped with thermometer, nitrogen inlet, and mechanical stirrer was charged N-(4-fluorophenyl)-N-(4-((7-methoxy-6-(methylcarbamoyl)quinolin-4-yl)oxy)phenyl)cyclopropane-1,1-dicarboxamide (1, 500 g; 1.0 eq.). The fumaric acid solution was clarified through a filter paper at 40-45° C., and transferred, at 40-45° C., to the flask with Compound 1. The 2000 mL round bottom flask was rinsed forward with 300 mL of a 20% solution of water in ethanol at 45-50° C. The resulting mixture was heated to reflux (75-80° C.) and stirred for 4-6 hours. The reaction mixture was then cooled to room temperature, and the product was filtered and the filter cake was washed twice with 300 mL of a 20% solution of water in ethanol. The product was then dried on a filter paper at room temperature or in a vacuum oven at 40-45° C. to yield a white to beige solid (472-474 g; 97%).

### Synthesis of N-(4-fluorophenyl)-N-(4-((7-methoxy-6-(methylcarbamoyl)quinolin-4-yl)oxy)phenyl)cyclopropane-1,1-dicarboxamide·1/2 fumaric acid (1·hemifumarate)—Method 2

Fumaric acid (2.68 g, 1 eq.) and EtOH/acetone, 1:1 (48 mL) were added to a two-piece EasyMax® (EM) (Mettler, Toledo, OH, USA) reaction vessel, and heated to a reaction temperature of 50° C. to dissolve all material. In the adjacent EM pot, a 1-piece EM vessel containing Compound 1 (12.0 g, 1 eq.) was set to a jacket temperature of 50° C. The fumaric acid solution was transferred to the vessel containing Compound 1. Seed was charged (2% seed, 0.244 g), and the vessel was heated to reflux (about 65° C.). After 1 hour, 0.5 mL of the slurry was filtered, washed with EtOH (6×1.5 mL) and analysed by HPLC to determine fumaric acid content (result should be about 10%). The slurry was then cooled to 25° C. over 1 hour and stirred for a further 1 hour. The solids were then filtered, washed with 1:1 EtOH/acetone (2×3 V), and dried over the weekend at 25° C. under vacuum. ¹H NMR 700 MHz (DMSO-d₆) δ 1.473 (s, 4H), δ 4.009 (s, 3H), δ 2.839 (d, 3H), δ 2.840 (d, 3H), δ 6.450 (d, 1H), δ 6.632 (s, 2H), δ 6.635 (s, 2H), δ 7.137 (m, 2H), δ 7.244 (d, 2H), δ 7.494 (s, 1H), δ 7.642 (m, 2H), δ 7.776 (d, 2H), δ 8.361 (q, 1H), δ 8.618 (s, 1H), 8.615 (s, 1H), δ 8.638 (d, 1H), δ 10.070 (s, 1H), δ 10.216 (s, 1H), δ 13.164 (s, 1H). ¹⁹F NMR 700 MHz (DMSO-d₆; ref. trifluorotoluene at −63.72 ppm) δ −121.460. ¹³C NMR 700 MHz (DMSO-d₆) δ 15.46, δ 26.47, δ 31.60, δ 56.15, δ 102.91, δ 107.83, δ 114.55, δ 115.05 (d), δ 121.15, δ 122.23, δ 122.43 (d), δ 124.35, δ 125.24, δ 134.03, δ 135.22 (d), δ 136.73, δ 149.08, δ 151.46, δ 153.18, δ 157.94, δ 158.30 (d), δ 161.76, δ 164.89, δ 168.16, and δ 168.16. ¹⁵N NMR 700 MHz (DMSO-d₆) δ 106.25 (¹⁵N), δ 127.79 (¹⁵N), δ 128.86 (¹⁵N), δ 166.04, 6 289.56 (¹⁵N).

### Compound 1 Hemifumarate Preparative Example 2

### Synthesis of 4-(4-aminophenoxy)-7-methoxy-N-methylquinoline-6-carboxamide

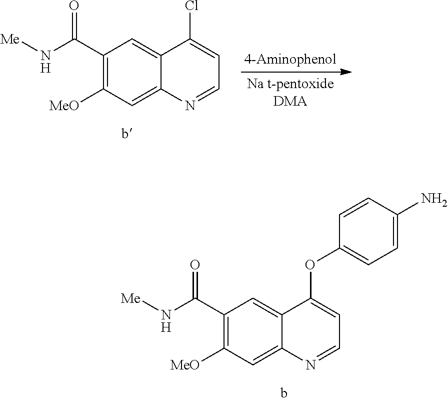 [SMILES: CNC(=O)c1cc2c(Cl)ccnc2cc1OC ; CNC(=O)c1cc2c(Oc3ccc(N)cc3)ccnc2cc1OC]

A reactor was charged with DMA (7.05 kg), 4-aminophenol (0.98 kg), and 4-chloro-7-methoxy-N-methylquinoline-6-carboxamide (b′; 1.5 kg). With agitation at room temperature, the batch was charged with 35% wt sodium t-pentoxide (2.635 kg). The reaction mixture was heated to 75-80° C. for 1 hour and cooled to 40-45° C. At 40-45° C., the reaction mixture was charged with water (15 kg). The batch was adjusted to 20-25° C., and agitated at for 1 h. The product, compound b, was filtered and washed with a mixture of DMA:water (2.8:3 kg) followed by water (6 kg) The product was dried on the filter for 66 h (shut down point). The procedure afforded 1.785 kg of compound b (92% yield).

### Synthesis of 1-((4-fluorophenyl)carbamoyl)cyclopropane-1-carbonyl chloride

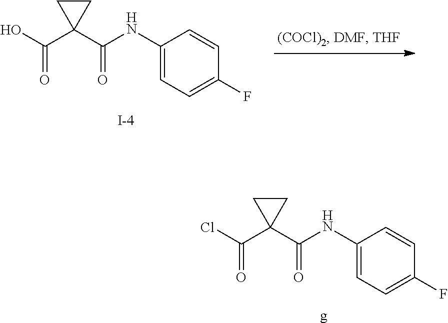

To a first reactor was charged carboxylic acid I-4 (1.6 kg), DMF (0.02 kg) and THF (4.6 kg). The acid dissolved and the solution was cooled to 5-10° C. To the solution was charged oxalyl chloride (0.898 kg) over 1.5 hour while keeping the temperature <20° C. The mixture was then warmed to 20-25° C. and agitation continued for 3 hours.

### Synthesis of N-(4-fluorophenyl)-N-(4-((7-methoxy-6-(methylcarbamoyl)quinolin-4-yl)oxy)phenyl)cyclopropane-1,1-dicarboxamide (1)

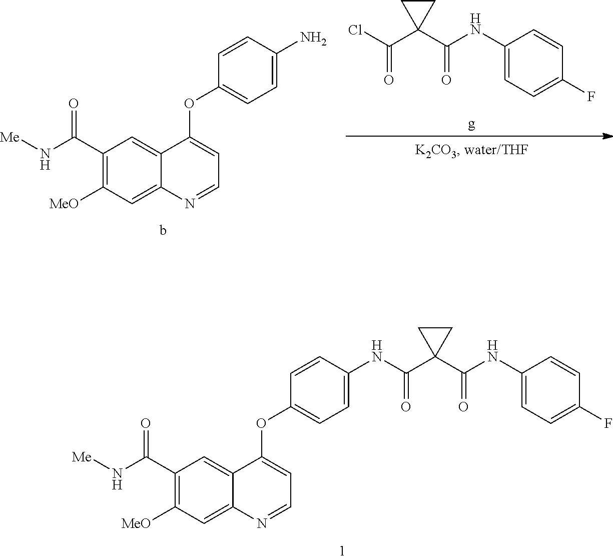 [SMILES: CNC(=O)c1cc2c(Oc3ccc(N)cc3)ccnc2cc1OC ; O=C(Cl)C1(C(=O)Nc2ccc(F)cc2)CC1 ; CNC(=O)c1cc2c(Oc3ccc(NC(=O)C4(C(=O)Nc5ccc(F)cc5)CC4)cc3)ccnc2cc1OC]

(i) To a second reactor was charged Compound b (1.759 kg) and THF (15.9 kg). To the mixture was charged a solution of K₂CO₃ (2.29 kg) in water (8.4 L), rinsed forward with water (0.5 kg). The contents of the first reactor were transferred to the second reactor over 40 min while keeping the temperature in the second reactor at 20-25° C., and the first reactor was rinsed forward with THF (2.4 kg). The mixture was agitated at 20-25° C. for 1 hour. The mixture in the second reactor was warmed to 35-40° C., the phases separated, and the lower aqueous phase was discarded.

(ii) The organic phase was warmed to 55-60° C. to dissolve solids in the mixture. The batch was clarified, transferred to the first reactor, and rinsed forward with THF (1.6 kg). To the first reactor was charged water (58 kg) over 1 hour. The batch was then agitated at 20-25° C. for 14 hours. The product was filtered and washed with a mixture of THE water (2.4.7 kg) and dried on the filter for a total of around 70 hours. The procedure provided 3.243 kg of compound 1 with a yield of 97.5%.

### Synthesis of N-(4-fluorophenyl)-N-(4-((7-methoxy-6-(methylcarbamoyl)quinolin-4-yl)oxy)phenyl)cyclopropane-1,1-dicarboxamide·1/2 fumaric acid (1·hemifumarate)

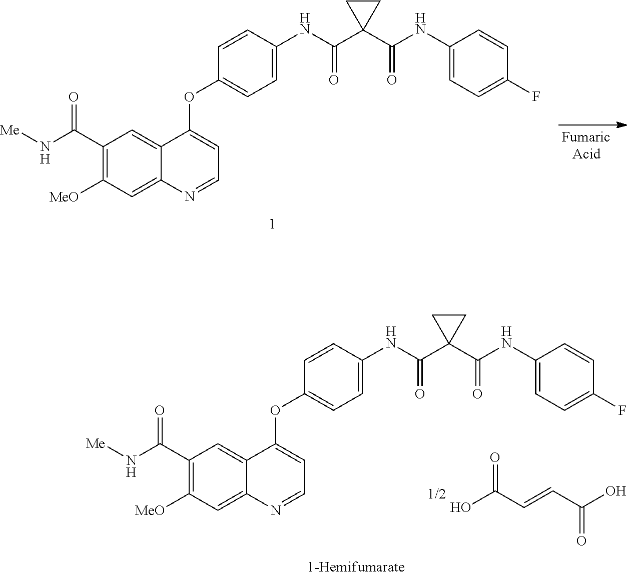 [SMILES: CNC(=O)c1cc2c(Oc3ccc(NC(=O)C4(C(=O)Nc5ccc(F)cc5)CC4)cc3)ccnc2cc1OC ; CNC(=O)c1cc2c(Oc3ccc(NC(=O)C4(C(=O)Nc5ccc(F)cc5)CC4)cc3)ccnc2cc1OC ; O=C(O)/C=C/C(=O)O]

To a third reactor was charged fumaric acid (0.58 kg), EtOH (4.9 kg) and water (1.5 kg). The batch was heated to 45-50° C. to dissolve the solids. To a fourth reactor was charged freebase compound 1 (3.23 kg) and the jacket temperature was adjusted to ca. 50° C. The contents in the third reactor was charged to the fourth reactor and rinsed forward with the mixture of water:EtOH (0.4:1.2 kg). The resulting mixture was heated to 75-80° C. and agitated for 4 h. The reaction was cooled to 45-50° C. and sampled for analysis. The mixture was further cooled to 20-25° C. and agitated for 1 hour. The product was filtered and washed with water:EtOH (0.8:2.4 kg). The product was dried on filter for 48 hours. The procedure provided 2.649 kg of compound 1 hemifumarate (74% yield).

### Compound 1 Hemifumarate Preparative Example 3

### Synthesis of 4-(4-aminophenoxy)-7-methoxy-N-methylquinoline-6-carboxamide

The procedure was conducted as described in Preparative Example 2.

### Synthesis of N-(4-fluorophenyl)-N-(4-((7-methoxy-6-(methylcarbamoyl)quinolin-4-yl)oxy)phenyl)cyclopropane-1,1-dicarboxamide (1)

The procedure was similar to Preparative Example 2, with the exception that the reaction temperature in the synthesis of Compound g was controlled at about 10-15° C. instead of 20-25° C. The reaction temperature in the synthesis of Compound 1, Step (i) was controlled at about 10-15° C. before warming to 35-40° C. The procedure provided 2.571 kg of freebase compound 1 (94% yield, when correcting for the presence of residual THF).

### Synthesis of N-(4-fluorophenyl)-N-(4-((7-methoxy-6-(methylcarbamoyl)quinolin-4-yl)oxy)phenyl)cyclopropane-1,1-dicarboxamide·1/2 fumaric acid (1·hemifumarate)

The procedure was conducted as described in Preparative Example 2, using 2.571 kg of freebase compound 1. The procedure provided 2.304 kg of compound 1 hemifumarate (88% yield)

### Compound 1 Hemifumarate Preparative Example 4

### Synthesis of 4-(4-aminophenoxy)-7-methoxy-N-methylquinoline-6-carboxamide

The procedure was conducted as described in Preparative Example 2.

### Synthesis of N-(4-fluorophenyl)-N-(4-((7-methoxy-6-(methylcarbamoyl)quinolin-4-yl)oxy)phenyl)cyclopropane-1,1-dicarboxamide (1)

The step (i) of the procedure was similar to step (i) of Preparative Example 2, using 1.5 kg of compound b, with the exception that the reaction temperature in the synthesis of Compound g was controlled at about 10-15° C. instead of 20-25° C. The reaction temperature in the synthesis of Compound 1, Step (i) was controlled at about 10-15° C. before warming to 35-40° C.

In step (ii), the organic phase was warmed to 55-60° C. and charged with water (9.7 kg) over 0.5 h. The mixture was seeded with solid Compound 1 (10 g) in water (0.1 kg) and agitated for 1 h 20 min. Additional water (26.7 kg) was introduced over 4 hr. The batch was cooled to 20-25° C. over 3 hours and agitated for 2.5 hours. The resulting product was filtered, washed with a mixture of THE (2.9 kg) and water (2.6 kg) and dried on filter for 80 hours. The procedure provided 2.054 kg of freebase Compound 1 (84% yield).

### Synthesis of N-(4-fluorophenyl)-N-(4-((7-methoxy-6-(methylcarbamoyl)quinolin-4-yl)oxy)phenyl)cyclopropane-1,1-dicarboxamide·1/2 fumaric acid (1·hemifumarate)

The procedure was conducted as described in Preparative Example 2, using 1.1794 kg of freebase compound 1. 5% Water in ethanol was used for the salt formation. The procedure provided 1.806 kg of compound 1 hemifumarate (92.4% yield).

### Analytical Examples

Compounds disclosed herein including Compound 1 and the impurities, contaminants, or degradants were analyzed through analytical procedures including but not limited to the following methods.

### Assay and Purity by High Performance Liquid Chromatography (UPLC)

Compound 1 hemifumarate FTIR sample was prepared by mixing approximately 6-7 mg of sample with approximately 200 mg of potassium bromide and placing in a sample cell holder. The spectrum was recorded over the range of 650 to 3800 cm¹. The identity of Compound 1 hemifumarate was confirmed if the FTIR spectrum of the sample conforms to that of the reference standard.

The assay of Compound 1 hemifumarate and determination of levels of impurities by weight percent or area percent was determined using a gradient reversed-phase UPLC method with 15% Acetonitrile/85% Ammonium Acetate Buffer, pH 8.0 as mobile phase A, and 80% Acetonitrile/20% Ammonium Acetate Buffer, pH 8.0 as mobile phase B. UV detection was conducted at 245 nm. Weight percent assay (% w/w free base and salt basis) was determined with a single point external reference standard. Area percent purity was determined by total area normalization.

### Impurities by LC-MS

The impurities or contaminants were determined by a gradient reverse-phase HPLC method coupled with QDa mass spectrometry (MS) detection. The levels of impurities are calculated against external reference standards.

### 4-Aminophenol by HPLC

The amount of 4-aminophenol in Compound 1 hemifumarate was determined using an ion exchange/reversed-phase mixed-mode HPLC method with UV detection at 192 nm. Mobile phase A was 30% Acetonitrile/70% Water with 0.3% phosphoric acid, and mobile phase B was 2% phosphoric acid in 100% Acetonitrile. The level of 4-aminophenol is calculated against the average response factor of three levels (5, 10, 15 ppm) of external standard concentration.

Table 2 below provides batch analysis for Compound 1 hemifumarate. Table 2 shows that by lowering the reaction temperature in the synthesis of Compound g from 20-25° C. to about 10-15° C., the levels of 4-aminophenol and 4-fluoroaniline in Compound 1 hemifumarate were reduced significantly. By seeding with Compound 1 and adding water slowly in Step (ii) of the synthesis of Compound 1, the level of 4-fluoroaniline in the final product was further reduced, leading to Compound 1 hemifumarate with minimum amount of impurities.

TABLE 2
Batch Analysis for Compound 1 Hemifumarate

| Impurity | Batch | Batch | Batch | Batch | Batch | Batch | Batch | Batch | Batch | Batch |
|---|---|---|---|---|---|---|---|---|---|---|
| (ppm) | 1¹ | 2² | 3³ | 4³ | 5³ | 6³ | 7³ | 8³ | 9³ | 10³ |
| 4-aminophenol | <3 | ND | ND | ND | ND | ND | ND | ND | ND | ND |
| 4-fluoroaniline | 5 | 3 | <1 | <1 | 2 | <1 | <1 | <2 | <2 | <2 |
| Compound a | NP | NP | <3 | 3 | <3 | <3 | <3 | <3 | <3 | <3 |
| Compound b | NP | NP | 68 | 39 | 36 | 31 | 16 | 36 | 31 | 16 |

¹Batch 1 was conducted using the process described in Preparative Example 2, which involves a reaction temperature of 20-25° C. in the synthesis of Compound g.
²Batch 2 was conducted using the process described in Preparative Example 3, which involves a reaction temperature of 10-15° C. in the synthesis of Compound g.
³Batches 3-10 were conducted using a process described in or similar to Example 1 or Example 4, which involves a reaction temperature of 10-15° C. in the synthesis of Compound g and a seeding step in the synthesis of Compound 1.
ND: Not Detected.
NP: Not Performed.

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1. Compound 1 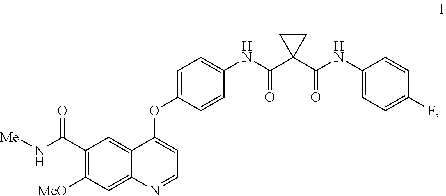 [SMILES: CNC(=O)c1cc2c(Oc3ccc(NC(=O)C4(C(=O)Nc5ccc(F)cc5)CC4)cc3)ccnc2cc1OC]
or a pharmaceutically acceptable salt thereof, comprising 200 ppm or less of contaminants or degradants.

2. The compound of claim 1, wherein the contaminants or degradants comprise 4-aminophenol, 4-fluoroaniline, 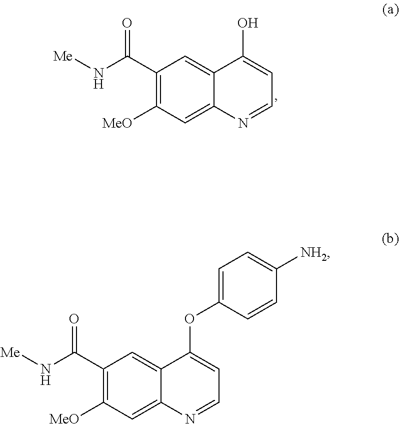 [SMILES: CNC(=O)c1cc2c(O)ccnc2cc1OC ; CNC(=O)c1cc2c(Oc3ccc(N)cc3)ccnc2cc1OC]
or mixtures thereof.

3. Compound 1 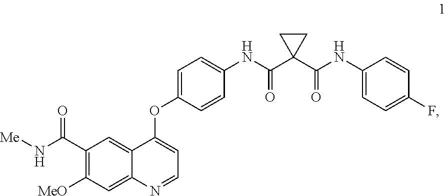 [SMILES: CNC(=O)c1cc2c(Oc3ccc(NC(=O)C4(C(=O)Nc5ccc(F)cc5)CC4)cc3)ccnc2cc1OC]
or a pharmaceutically acceptable salt thereof, comprising 100 ppm or less of genotoxic contaminants or degradants.

4. The compound of claim 3, wherein the genotoxic contaminants or degradants comprise 4-aminophenol, 4-fluoroaniline, 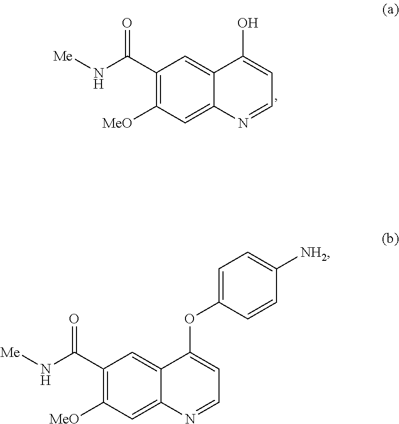 [SMILES: CNC(=O)c1cc2c(O)ccnc2cc1OC ; CNC(=O)c1cc2c(Oc3ccc(N)cc3)ccnc2cc1OC]
or mixtures thereof.

5. The compound of any one of claims 1-4, wherein the contaminants or degradants are 100 ppm or less, 75 ppm or less, 50 ppm or less, 25 ppm or less, 20 ppm or less, 15 ppm or less, 13 ppm or less, 10 ppm or less, 5 ppm or less, 3 ppm or less, 2.5 ppm or less, or 1 ppm or less.

6. The compound of any one of claims 1, 2, and 5, wherein the contaminants or degradants comprise genotoxic contaminants or degradants.

7. The compound of claim 6, wherein the genotoxic contaminants or degradants comprise 4-aminophenol, 4-fluoroaniline, or mixtures thereof.

8. The compound of any one of claims 1-7, wherein the pharmaceutically acceptable salt is Compound 1 hemifumarate.

9. The compound of any one the proceeding claims, wherein the contaminants or degradants comprise 4-aminophenol.

10. The compound of claim 9, wherein Compound 1 or Compound 1 hemifumarate is mixed with 15 ppm or less, 10 ppm or less, 5 ppm or less, 3 ppm or less, or 1 ppm or less 4-aminophenol.

11. The compound of any one of the proceeding claims, wherein the contaminants or degradants comprise 4-fluoroaniline.

12. The compound of claim 11, wherein Compound 1 or Compound 1 hemifumarate is mixed with 15 ppm or less, 10 ppm or less, 5 ppm or less, 3 ppm or less, 2 ppm or less, or 1 ppm or less 4-fluoroaniline.

13. The compound of claim 12, wherein Compound 1 or Compound 1 hemifumarate is mixed with 5 ppm or less 4-fluoroaniline.

14. The compound of claim 12, wherein Compound 1 or Compound 1 hemifumarate is mixed with 3 ppm or less 4-fluoroaniline.

15. The compound of claim 12, wherein Compound 1 or Compound 1 hemifumarate is mixed with 2 ppm or less 4-fluoroaniline.

16. The compound of claim 12, wherein Compound 1 or Compound 1 hemifumarate is mixed with 1 ppm or less 4-fluoroaniline.

17. The compound of any one of the proceeding claims, wherein the contaminants or degradants comprise Compound a.

18. The compound of claim 17, wherein Compound 1 or Compound 1 hemifumarate is mixed with 15 ppm or less, 10 ppm or less, 5 ppm or less, or 3 ppm or less Compound a.

19. The compound of any one of the proceeding claims, wherein the contaminants or degradants comprise Compound b.

20. The compound of claim 19, wherein Compound 1 or Compound 1 hemifumarate is mixed with 100 ppm or less, 75 ppm or less, 50 ppm or less, 25 ppm or less, 15 ppm or less, 10 ppm or less, 5 ppm or less, or 3 ppm or less Compound b.

21. A pharmaceutical composition comprising Compound 1: 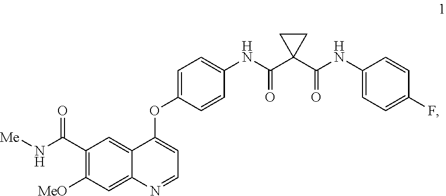 [SMILES: CNC(=O)c1cc2c(Oc3ccc(NC(=O)C4(C(=O)Nc5ccc(F)cc5)CC4)cc3)ccnc2cc1OC]
or a pharmaceutically acceptable salt thereof, and one or more pharmaceutically acceptable excipients, wherein the pharmaceutical composition comprises 200 ppm or less of contaminants or degradants.

22. The pharmaceutical composition of claim 21, wherein the contaminants or degradants are 100 ppm or less, 75 ppm or less, 50 ppm or less, 25 ppm or less, 20 ppm or less, 15 ppm or less, 13 ppm or less, 10 ppm or less, 5 ppm or less, 2.5 ppm or less, or 1 ppm or less.

23. The pharmaceutical composition of claim 21 or 22, wherein the contaminants or degradants comprise 4-aminophenol, 4-fluoroaniline, 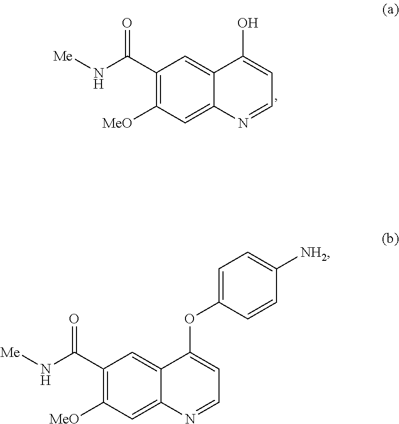 [SMILES: CNC(=O)c1cc2c(O)ccnc2cc1OC ; CNC(=O)c1cc2c(Oc3ccc(N)cc3)ccnc2cc1OC]
or mixtures thereof.
